SEQUENCE LISTING (4 sequences)

SEQ ID NO: 1 - protein, 123 aa, Artificial Sequence
  Synthetic construct
QVQLVQSGAEVKKPGASVKVSCKASGYTFTSYRIHWVRQAPGQGLEWMGEIYPSNARTNYNEKFKSRVTMTRDTSTSTVYMELSSLRSEDTAVYYCARKYYYGNTRRSWYFDVWGQGTTVTVS

SEQ ID NO: 2 - protein, 113 aa, Artificial Sequence
  Synthetic construct
DIQMTQSPSSLSASVGDRVTITCRASESIDNYGISFLAWYQQKPGKAPKLLIYAASNRGSGVPSRFSGSGSGTDFTFTISSLQPEDIATYYCQQSKTVPRTFGQGTKLEIKRT

SEQ ID NO: 3 - DNA, 21 bp, Artificial Sequence
  Synthetic construct
gctggaaacgatcacataccg

SEQ ID NO: 4 - DNA, 20 bp, Artificial Sequence
  Synthetic construct
agctcctgatgatccaatgg

(19) United States
(12) Patent Application Publication
(10) Pub. No.: US 2007/0031418 A1
(43) Pub. Date: Feb. 8, 2007

(54) Methods for treating lower motor neuron diseases and compositions containing the same

(76) Inventors: Lucia Tabares, Sevilla (ES); Arnon Rosenthal, Woodside, CA (US); John C. Lin, Mountain View, CA (US)

Correspondence Address: MORRISON & FOERSTER LLP, 755 PAGE MILL RD, PALO ALTO, CA, 94304-1018, US

(21) Appl. No.: 11/412,685

(22) Filed: Apr. 26, 2006

Related U.S. Application Data
(60) Provisional application No. 60/675,393, filed on Apr. 26, 2005.

(51) Int. Cl. A61K 39/395

(52) U.S. Cl. 424/146.1

(57) ABSTRACT

This invention provides methods for the treatment, prevention, and/or amelioration of symptoms relating to lower motor neuron diseases (such as spinal muscular atrophy). The methods comprise administration of an agonist anti-trkC antibody. Compositions and kits are also provided.

### CROSS-REFERENCE TO RELATED APPLICATIONS

This application claims the priority benefit of the provisional patent application U.S. Ser. No. 60/675,393, filed Apr. 26, 2005, which is incorporated herein by reference in its entirety.

### STATEMENT REGARDING FEDERALLY SPONSORED RESEARCH OR DEVELOPMENT

Not applicable.

### FIELD OF THE INVENTION

The invention concerns use of agonist anti-trkC antibodies in the treatment and/or prevention of lower motor neuron diseases.

### BACKGROUND OF THE INVENTION

Motor neuron disease is a disorder in which motor neurons degenerate and die. Motor neurons, including upper motor neurons and lower motor neurons, affect voluntary muscles, stimulating them to contract. Upper motor neurons originate in the cerebral cortex and send fibers through the brainstem and the spinal cord, and are involved in controlling lower motor neurons. Lower motor neurons are located in the brainstem and the spinal cord and send fibers out to muscles. Lower motor neuron diseases are diseases involving lower motor neuron degeneration. When a lower motor neuron degenerates, the muscle fibers it normally activates become disconnected and do not contract, causing muscle weakness and diminished reflexes. Loss of either type of neurons results in weakness, muscle atrophy (wasting) and painless weakness are the clinical hallmarks of motor neuron disease.

SMARD1 is a clinical variant of Spinal Muscular Atrophy (SMA), the second most common autosomal recessive disorder, and the most common genetic cause of death in childhood. SMA and SMARD1 are characterized by degeneration of lower motor neurons associated with progressive muscle paralysis. While the majority of SMA cases is due to mutations in the Survival Motor Neuron gene (SMN), (1) SMARD1 is caused by mutations in a different gene, the immunoglobulin μ-binding protein 2 gene (IGHMBP2) (2-5). SMARD1 patients suffer from early impairment of the respiratory function due to diaphragmatic involvement (4). Currently, there is no effective therapy for either SMARD1 or SMA in general (6), despite some pilot clinical trials with positive results (7).

The nmd mice harbour a spontaneous mutation in the mouse Ighmbp2 gene, a member of a DNA/RNA helicase/ATPase protein family (8-10). The genetic defect consists of a single mutation (A to G) in intron 4, resulting in 80% abnormally spliced and 20% full length transcript (9). The nmd mice display a disease phenotype similar to the milder form of human SMARD1 as the functional IGHMBP2 expression is not completely abolished in the nmd mice (9). Muscular weakness starts to develop after the second week of birth, progressing to severe neurogenic muscle atrophy of the extremities (8-11).

Neurotrophic factors have been considered as potential therapeutics for motor neurons diseases. This expectation has been based on the survival-promoting properties of these molecules in animal embryonic motor neurons in culture, their positive biological effects on nerves after axotomy and on alleviating the pathological symptoms in animal models of neurodegenerative diseases (12-15). Given the promising results obtained in most of the in vitro and in vivo studies, exogenous neurotrophins has been used in clinical trials for patients with Alzheimer disease, amyotrophic lateral sclerosis (ALS), peripheral neuropathies, Parkinson's and Hungtinton's disease.

One practical difficulty in applying neurotrophins is that these proteins all have a relatively short half life while the neurodegenerative diseases are chronic and require long term treatment. Therapeutic agonist antibodies targeting the neurotrophin receptors may represent a novel approach for neurodegenerative diseases due to their high specificity and long half life.

All references cited herein, including patent applications and publications, are incorporated by reference in their entirety.

### BRIEF SUMMARY OF THE INVENTION

The invention provides methods for treating lower motor neuron diseases (involving lower motor neuron degeneration) in an individual. Examples of lower motor neuron diseases are spinal muscular atrophy (SMA), and spinal muscular atrophy with respiratory distress type 1 (SMARD1). The methods comprise administering to the individual an effective amount of an agonist anti-trkC antibody.

In one aspect, the invention provides methods for treating a lower motor neuron disease in an individual, comprising administering to the individual an effective amount of an agonist anti-trkC antibody. In another aspect, the invention provides methods of delaying development of a symptom associated with a lower motor neuron disease in an individual comprising administering to the individual an effective amount of an agonist anti-trkC antibody. In another aspect, the invention provides methods of ameliorating a symptom of a lower motor neuron disease in an individual comprising administering to the individual an effective amount of an agonist anti-trkC antibody.

In some embodiments, the muscle strength in the individual is improved after the administration of the agonist anti-trkC antibody. In some embodiments, the decline of muscle strength in the individual is delayed after administration of the agonist anti-trkC antibody.

In some embodiments, the individual is a mammal, such as a human.

Agonist anti-trkC antibodies are known in the art. In some embodiments, the agonist anti-trkC antibody is a monoclonal antibody. In some embodiments, the agonist anti-trkC antibody binds human trkC. In some embodiments, the agonist anti-trkC antibody specifically binds human trkC. The agonist anti-trkC antibody may also bind human and rodent trkC. The agonist anti-trkC antibody may be a human antibody (such as antibody 6.4.1 (PCT Publication No. WO 01/98361)) or may be a humanized antibody (including humanized monoclonal antibody 2256). In another embodiment, the agonist anti-trkC antibody is humanized antibody A5, as described in PCT WO2004/058190 and herein. In still other embodiments, the anti-trkC agonist antibody comprises the amino acid sequence of the heavy chain variable region shown in Table 1 (SEQ ID NO:1) and the amino acid sequence of the light chain variable region shown in Table 2 (SEQ ID NO:2). In other embodiments, the anti-trkC agonist antibody comprises one or more CDR(s) of antibody A5 (such as one, two three, four, five or, in some embodiments, all six CDRs from A5). Identification of CDRs is well within the skill of the art. In some embodiments, the CDRs comprise the Kabat CDR. In other embodiments, the CDRs are the Chothia CDR. In still other embodiments, the CDR comprises both the Kabat and Chothia CDRs. In some embodiments, the antibody comprises a light chain that is encoded by a polynucleotide in a vector with a deposit number of ATCC No. PTA-5682. In some embodiments, the antibody comprises a heavy chain that is encoded by a polynucleotide in a vector with a deposit number of ATCC No. PTA-5683. In some embodiments, the antibody comprises (a) a light chain that is encoded by a polynucleotide in a vector with a deposit number of ATCC No. PTA-5682; and (b) a heavy chain that is encoded by a polynucleotide in a vector with a deposit number of ATCC No. PTA-5683. In some embodiments, the antibody comprises one or more CDR(s) encoded by (a) a polynucleotide in a vector with a deposit number of ATCC No. PTA-5682; and/or (b) a heavy chain that is encoded by a polynucleotide in a vector with a deposit number of ATCC No. PTA-5683.

The antibody may bind essentially the same trkC epitope as or compete for binding with an antibody selected from any one or more of the following: 6.1.2, 6.4.1, 2345, 2349, 2.5.1, 2344, 2248, 2250, 2253, and 2256. See PCT Publication No. WO 01/98361. The antibody may comprise a modified constant region, such as a constant region that is immunologically inert, e.g., does not trigger a complement mediated lysis or does not stimulate antibody-dependent cell mediated cytotoxicity (ADCC). In other embodiments, the constant region is modified as described in Eur. J. Immunol. (1999) 29:2613-2624; PCT Application No. PCT/GB99/01441; and/or UK Patent Application No. 9809951.8.

The antibody may also be an antibody fragment, such as an antibody fragment selected from one or more of the following: Fab, Fab′, F(ab′)₂, Fv fragments, diabodies, single chain antibody molecules and multispecific antibodies formed from antibody fragments, and a single-chain Fv (scFv) molecule. The antibody may also be chimeric, and it may be bispecific.

Administration of an agonist anti-trkC antibody can be by any suitable method known in the art, including one or more of the following means: intravenously, subcutaneously, via inhalation, intrarterially, intramuscularly, intracardially, intraventricularly, intrathecally, intraspinally, and intraperitoneally. Administration may be systemic (e.g. intravenously) and/or localized. Administration may be acute and/or chronic. Administration can be before onset of the lower motor neuron disease.

In another aspect, the invention provides compositions and kits comprising an agonist anti-trkC antibody for use in any of the methods of the invention. These kits may further comprise instructions for use of the agonist anti-trkC antibody in any of the methods described herein. The invention also provides pharmaceutical compositions comprising an agonist anti-trkC antibody and a pharmaceutically acceptable carrier for use any of the methods described herein.

The invention also provides any of the compositions and kits described for any use described herein whether in the context of use as medicament and/or use for manufacture of a medicament.

### BRIEF DESCRIPTION OF THE DRAWINGS

FIG. 1 shows that monoclonal antibody Mab2256 can activate the trkC receptor and support trigeminal neuronal survival in culture. (A) Increasing and saturable levels of trkC receptor phosphoryaltion (expressed in OD 450, the y axis) were induced either by increasing concentrations of NT3 (left panel), the endogenous trkC ligand, or by the monoclonal antibody Mab2256 (right panel), a trkC antibody. (B) Increasing and saturable numbers of embryonic rat trigeminal neurons surviving 48 hours in culture were supported by the presence of various concentrations of NT3 (left panel) or Mab2256 (right panel) in the culture medium.

FIG. 2 shows disease signs and lifespan in nmd mice. (A) Mean body weight of wild-type (+/+) (n=8), nmd (−/−) (n=8), and heterozygous (+/−)(n=8)mice from P20 to P70. (B) Mean body weight of wild-type (+/+ Ab) (n=10) and nmd mice (−/− Ab) (n=16) injected intraperitoneally with monoclonal antibody Mab2256, from week 3 to 11. (C-D) Fore limb grip time (C), and balance on the rod (D) of wild-type (+/+, open symbols) (n=14), heterozygous (+/−, grey symbols) (n=8), nmd injected with PBS (−/−, filled squares) (n=5), and nmd mice injected with Mab2256 (−/− Ab, triangles) (n=9). (E-F) Wild type and Mab2256-treated mutant on the rod. The mutant was not able to use its tail to grasp the rod; nevertheless the mouse was able to maintain itself on the rotating rod for several seconds. Mab2256-untreated nmd mice were not able to maintain themselves for more than one second on the rod (not shown). (G) Kaplan-Meier survival analysis of untreated (nmd) (n=30) and Mab2256-treated nmd (nmd Ab) (n=15) mice. No significant differences for the two groups were obtained with the Mann-Whitney Rank-Sum test.

FIG. 3 shows that EMG measurements of CMAP amplitudes in the medial gastrocnemius (MG) of wild-type and nmd mice reveal reduction of neurotransmission efficacy in the nmd mice. (A) Absolute amplitudes of CMAP (means±SEMs) in response to supramaximal stimulation in untreated wild-type (+/+) (n=14), nmd injected or not with PBS (−/−) (n=9), wild-type treated with Mab2256 (+/+ Ab) (n=8), nmd treated with Mab2256 (−/− Ab) (n=7) and nmd mice treated with NT-4/5 (−/− NT4/5) (n=3). (B-C) Representative responses of the CMAPs to a pair pulse protocol in a wild-type (B) and in an nmd mouse (C). Interstimulus interval: 10 ms. The peak-to-peak amplitudes of the successive CMAPs (A1 and A2) are signalled by arrows. (D) Representative recordings during a train of stimuli at 100 Hz in a wild-type (upper trace), and three nmd mice: untreated (second trace), treated with Mab2256 (third trace) and treated with NT-4/5 (forth trace). (E) Depression of CMAP amplitudes (normalized to the first response) during a train of stimuli of 250 ms at 100 Hz in untreated wild-type mice (+/+) (n=6), Mab2256-treated wild-type (+/+ Ab) (n=8), PBS injected nmd (−/−) (n=6), Mab2256-treated nmd (−/− Ab) mice (upper graph, n=6) and NT-4/5-treated mice (−/− NT4/5) (lower graph, n=3). (F) Percent of depression of the CMAP amplitudes at the quasi steady-state level for stimulation frequencies from 10-100 Hz. All data are from EMG recordings done at P69-71.

FIG. 4 shows EMG measurements of CMAP amplitudes in the dorsal foot muscles. (A) M and H-waves elicited by a single pulse stimulus in a wild-type mouse. (B) Percent of depression of the CMAP amplitudes at the quasi steady-state level for stimulation frequencies from 10-100 Hz in wild-type (+/+) (n=6) and nmd mice (−/−) (n=6). (C) Time course of the depression of the CMAP amplitudes (normalized to the first response) during a train of stimuli of 250 ms at 100 Hz in wild-type mice (n=6) and PBS injected nmd (n=6). All data are from mice at P69-71.

FIG. 5 shows motor unit number estimate (MUNE) from the MG in mice at P215-230. (A-B) Motor unit traces from wild-type (A) and nmd (B) mice. (C-F) Amplitudes of single motor unit action potentials (SMUAPs) in control (C & E) and nmd sib mutants (D & F) mice in response to stimuli of increasing amplitude. Each number in the X axis represents a stimulus that elicited an increment in the amplitude of the response. Control animals were heterozygous.

FIG. 6 shows spontaneous electrical activity in the diaphragm recorded in vivo. (A and B) Representative recordings from control (A) and nmd mice (B). Lower traces are raw recordings and upper traces the integral of lower traces. Circles signals electrical activity from the heart. (C) Histogram showing the mean duration of the inspiration bursts (TI), in ms, in control (white bar) and PBS-injected mutant (black bar) and Mab2256-treated mutant mice. (D) Histological sections of control and nmd phrenic nerves. (E) Quantification of the number of myelinated axons in four 38-weeks-old mice showing no significant differences between control and nmd littermates. Bars: 7 μm.

### DETAILED DESCRIPTION OF THE INVENTION

The present invention provides methods for treating a lower motor neuron disease in an individual, comprising administering to the individual an effective amount of an agonist anti-trkC antibody. Examples of lower motor neuron disease include SMA and a clinical variant of SMA, SMARD1.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture. Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### DEFINITIONS

An “antibody” (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, humanized antibodies, chimeric antibodies, diabodies linear antibodies, single chain antibodies, multispecific antibodies (e.g., bispecific antibodies) and any other modified configuration of the immunoglobulin molecule that comprises an antigen recognition site of the required specificity. An antibody includes an antibody of any class, such as IgG, IgA, or IgM, and the antibody need not be of any particular class. Depending on the antibody amino acid sequence of the constant domain of its heavy chains, immunoglobulins can be assigned to different classes. There are five major classes of immunoglobulins: IgA, IgG, IgD, IgE, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, gamma, delta, epsilon, and mu, respectively. There are also two classes of light chain, designated kappa and lambda. The subunit structures and three-dimensional configurations of different classes of immunoglobulins are well known.

A “monoclonal antibody” refers to a homogeneous antibody population wherein the monoclonal antibody is comprised of amino acids (naturally occurring and non-naturally occurring) that are involved in the selective binding of an antigen. A population of monoclonal antibodies is highly specific, being directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a trkC epitope is an antibody that binds this trkC epitope with greater affinity, avidity, more readily, and/or with greater duration than it binds to other trkC epitopes or non-trkC epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “functional Fc region” possesses at least one effector function of a native sequence Fc region. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

An “agonist anti-trkC antibody” (interchangeably termed “anti-trkC agonist antibody”) refers to an antibody that is able to bind to and activate a trkC receptor and/or downstream pathway(s) mediated by the trkC signaling function. For example, the agonist antibody may bind to the extracellular domain of a trkC receptor and thereby cause dimerization of the receptor, resulting in activation of the intracellular catalytic kinase domain. Consequently, this may result in stimulation of growth and/or differentiation of cells expressing the receptor in vitro and/or in vivo. In some embodiments, an agonist anti-trkC antibody binds to trkC and activates a trkC biological activity. In some embodiments, an agonist antibody useful in the methods of the invention recognizes domain V and/or domain IV of trkC. See Urfer et al., J. Biol. Chem. 273: 5829-5840 (1998).

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

As used herein, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

As used herein, “affinity matured” antibody means an antibody with one or more alterations in one or more CDRs thereof that result an improvement in the affinity of the antibody for antigen compared to a parent antibody that does not possess the alteration(s). In some embodiments, affinity matured antibodies will have nanomolar or even picomolar affinities for the target antigen. Affinity matured antibodies are produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al, 1992, J. Mol. Biol., 226:889-896).

As used herein, “trkC” refers to the trkC receptor polypeptide, a member of the tyrosine kinase superfamily. TrkC encompasses the native trkC receptor of any mammalian species, including but not limited to, human, canine, feline, bovine, equine, primate, and rodent (including mouse and rat). The extracellular domain of full-length native trkC has been defined with reference to homologous or otherwise similar structures identified in various other proteins. The domains have been designated starting at the N-terminus of the mature trkC receptor as: 1) a first cysteine-rich domain extending from amino acid 1 to amino acid 48; 2) a leucine-rich domain extending from amino acid 49 to amino acid 120; 3) a second cysteine-rich domain extending from amino acid 121 to amino acid 177; 4) a first immunoglobulin-like domain extending from about amino acid 196 to amino acid 257; and 5) a second immunoglobulin-like domain extending from about amino acid 288 to amino acid 351. See, e.g., PCT Publication No. WO 0198361. The domain structure of the human trkC receptor has also been designated by reference to a crystal structure as follows: domain 1 from amino acid 1 to amino acid 47; domain 2 from amino acid 48 to amino acid 130; domain 3 from amino acid 131 to amino acid 177; domain 4 from amino acid 178 to amino acid 165; and domain 5 from amino acid 166 to amino acid 381. See, e.g., PCT Publication No. WO 0198361; Urfer et al. J. Biol. Chem. 273: 5829-5840 (1998). Also included are variants of trkC, examples of which include, but are not limited to, variants without a kinase domain (Shelton, et al., J. Neurosci. 15(1):477-491 (1995)), and variants with a modified kinase domain (Shelton, et al., J. Neurosci. 15(1):477-491 (1995)).

“Biological activity”, when used in conjunction with the agonist anti-trkC antibodies of the present invention, generally refers to having the ability to bind and activate the trkC receptor tyrosine kinase and/or a downstream pathway mediated by the trkC signaling function. As used herein, “biological activity” encompasses one or more effector functions in common with those induced by action of NT-3, the native ligand of trkC, on a trkC-expressing cell. A “biological activity” of trkC can also encompass downstream signaling pathway(s) or effector functions that are different than those induced by action of NT-3. Without limitation, biological activities include any one or more of the following: ability to bind and activate trkC; ability to promote trkC receptor dimerization; the ability to promote the development, survival, function, maintenance and/or regeneration of cells (including damaged cells), in particular neurons in vitro or in vivo, including peripheral (sympathetic, sensory, and enteric) neurons, and central (brain and spinal cord) neurons, and non-neuronal cells, e.g. peripheral blood leukocytes. A particular preferred biological activity is the ability to treat (including prevention of) one or more symptoms of a lower motor neuron disease, and/or repair and/or improve the function of lower motor neurons.

As used herein, “treatment” is an approach for obtaining beneficial or desired clinical results. For purposes of this invention, beneficial or desired clinical results include, but are not limited to, one or more of the following: alleviation of one or more symptoms associated with lower motor neuron disease (e.g., degeneration of lower motor neurons, progressive muscle weakness or paralysis, respiratory failure, and diminishing reflexes); delaying or slowing of progression of the lower motor neuron disease; stabilizing (i.e., not worsening) lower motor neuron degeneration; amelioration of degeneration of lower motor neurons and/or weakness of muscles; delaying the need for wheel-chair use; delaying the need for artificial ventilation; and ultimately prolonging the overall survival.

“Palliating” a lower motor neuron disease or one or more symptoms of a lower motor neuron disease means lessening the extent and/or time course of undesirable clinical manifestations of the disease in an individual or population of individuals treated with an agonist anti-trkC antibody in accordance with the invention.

“Reducing severity of a symptom” or “ameliorating a symptom” of a lower motor neuron disease means a lessening and/or improvement of one or more symptoms of a lower motor neuron disease as compared to not administering an agonist anti-trkC antibody. “Reducing severity” also includes shortening or reduction in duration of a symptom.

As used herein, “delaying” development of a lower motor neuron disease means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the lower motor neuron degeneration and muscle weakness or paralysis. A method that “delays” development of a lower motor neuron disease is a method that reduces probability of development of the lower motor neuron degeneration in a given time frame and/or reduces extent of the lower motor neuron degeneration in a given time frame, when compared to not using the method. Such comparisons are typically based on clinical studies, using a statistically significant number of subjects.

“Development” of a lower motor neuron disease means the onset and/or progression of symptoms associated with the lower motor neuron disease (e.g., deterioration in lower motor neuron function, and muscle weakness and paralysis) within an individual. Development can be detectable using standard clinical techniques as described herein. However, development also refers to disease progression that may be initially undetectable. For purposes of this invention, progression refers to the biological course of the disease state, in this case, as determined by a standard neurological examination, or patient interview or may be determined by more specialized quantitative testing. These more specialized quantitative tests may include, but are not limited to, determination of conduction velocity of the affected neurons by means of microneurography, specialized tests of balance, tests of reflexes, specialized tests of proprioception, and/or kinesthetic sense, tests of strength (e.g., clinical examination of muscle strength, including, but not limited to, quantitative measures of muscle strength, electromyography, MRI) tests of autonomic function, including, but not limited to, test of blood pressure control, tests of heart rate response to various physiological and pharmacological stimuli. By way of example, for SMA, the tests may include tests of motor skill and/or strength, such as clinical examination of muscle strength, including, but not limited to, quantitative measures of muscle strength, electromyography and/or MRI. “Development” includes occurrence, recurrence, and onset. As used herein “onset” or “occurrence” of a lower motor neuron disease includes initial onset and and/or recurrence.

As used herein, an “at risk” individual is an individual who is at risk of development of a lower motor neuron disease. An individual “at risk” may or may not have detectable disease, and may or may not have displayed detectable disease prior to the treatment methods described herein. “At risk” denotes that an individual has one or more so-called risk factors, which are measurable parameters that correlate with development of the disease, such as mutations in the survival motor neuron gene (SMN) or the immunoglobulin μ-binding protein 2 gene (IGHMBP2). An individual having one or more of these risk factors has a higher probability of developing a lower motor neuron disease than an individual without these risk factor(s).

An “effective amount” is an amount sufficient to effect beneficial or desired clinical results including clinical results or delaying the onset of the disease. An effective amount can be administered in one or more administrations. For purposes of this invention, an effective amount of an agonist anti-trkC antibody described herein is an amount sufficient to ameliorate, stabilize, reverse, slow and/or delay progression of or prevent a lower motor neuron disease. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and single agent may be considered to be given in an effective amount if, in conjunction with one or more other agents, a desirable result may be or is achieved.

As used herein, administration “in conjunction” includes simultaneous administration and/or administration at different times. Administration in conjunction also encompasses administration as a co-formulation or administration as separate compositions. As used herein, administration in conjunction is meant to encompass any circumstance wherein an agonist anti-trkC antibody and another therapeutic agent for the lower motor neuron disease or combination thereof are administered to an individual, which can occur simultaneously and/or separately. As further discussed herein, it is understood that an agonist anti-trkC antibody and the other therapeutic agent can be administered at different dosing frequencies or intervals. It is understood that the agonist anti-trkC antibody and the other therapeutic agent can be administered using the same route of administration or different routes of administration.

An “individual” is a mammal, more preferably a human. Mammals also include, but are not limited to, farm animals, sport animals, pets, primates, horses, cows, cats, dogs, and rodents (such as mice and rats).

As used herein, the singular form “a”, “an”, and “the” includes plural references unless indicated otherwise. For example, “an” antibody includes one or more antibodies and “a symptom” means one or more symptoms.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

Methods of the Invention

With respect to all methods described herein, reference to agonist anti-trkC antibodies also includes compositions comprising one or more of these antibodies. These compositions may further comprise suitable excipients, such as pharmaceutically acceptable excipients including buffers, which are well known in the art.

Methods of Treating Lower Motor Neuron Diseases Using Agonist Anti-trkC Antibodies

The present invention encompasses methods of treating, preventing, delaying the development of a symptom of and/or palliating a lower motor neuron disease using agonist anti-trkC antibodies. The methods entail administering an effective amount of these antibodies to an individual in need thereof (various indications and aspects are described herein). An effective amount of the agonist anti-trkC antibody may be administered with or without other therapeutic agents. The individual may have been diagnosed with the lower motor neuron disease or may be at risk of developing the lower motor neuron disease. In some embodiments, the individual is human. However, the methods described are also applicable to the veterinary context (e.g. non-human mammal, such as dogs, cats, cattle, horses).

Methods of assessing lower motor neuron diseases, such as SMA and SMARD1, and treatment thereof are known in the art and described herein.

Agonist Anti-trkC Antibodies

Methods of the invention entail using anti-trkC antibodies that interact with trkC in a manner that activates trkC. An anti-trkC agonist antibody should exhibit any one or more of the following characteristics: (a) bind to trkC receptor; (b) bind to one or more epitopes of trkC receptor; (c) bind to trkC receptor and activate trkC biological activity(ies) or one or more downstream pathways mediated by trkC signaling function(s); (d) promote trkC receptor dimerization; (e) increase trkC receptor activation; (f) display favorable pharmacokinetic and bioavailability properties; (g) promote the development, survival, function, maintenance and/or regeneration of cells; (h) bind to trkC receptor and treat, prevent, reverse, or ameliorate one or more symptoms of a lower motor neuron disease.

Agonist anti-trkC antibodies are known in the art. See PCT Publication No. WO 01/98361; Urfer et al. J. Biol. Chem. (1998)273:5829-5840. In some embodiments, the anti-trkC agonist antibody is a humanized mouse anti-trkC agonist antibody termed antibody “A5”, which comprises the human heavy chain IgG2a constant region containing the following mutations: A330P331 to S330S331 (amino acid numbering is based on Kabat numbering with reference to the wildtype IgG2a sequence; see Eur. J. Immunol. (1999) 29:2613-2624); the human light chain kappa constant region; and the heavy chain and light chain variable regions shown in Tables 1 and 2.

TABLE 1

A5 heavy chain variable region. Chothia CDRs are shown as underlined italics;
Kabat CDRs are shown in bold.

| | |
|---|---|
| QVQLVQSGAEVKKPGASVKVSCKASGYTFTSYRIHWVRQAPGQGLEWMG [symbol] | (SEQ ID NO: 1) |
| [symbol] SRVTMTRDTSTSTVYMELSSLRSEDTAVYYCAR [symbol] WGQGTTVTVS | |

TABLE 2

A5 light chain variable region. Kabat CDRs are shown as underlined italics;
Chothia
CDRs are shown as bold.

| | |
|---|---|
| DIQMTQSPSSLSASVGDRVTITC [symbol] WYQQKPGKAPKLLIY [symbol] | (SEQ ID NO: 2) |
| [symbol] GVPSRFSGSGSGTDFTFTISSLQPEDIATYYC [symbol] FGQGTKLEIKRT | |

The following polynucleotides encoding the heavy chain variable region or the light chain variable region of A5 were deposited with the American Type Culture Collection, 10801 University Boulevard, Manassas, Va., USA (ATCC):

| | | ATCC | |
|---|---|---|---|
| Material | | Accession No. | Date of Deposit |
| Eb.pur.2256.A5 | A5 light chain | PTA-5682 | Dec. 5, 2003 |
| Db.2256.A5 | A5 heavy chain | PTA-5683 | Dec. 5, 2003 |

Vector Eb.pur.2256.A5 is a polynucleotide encoding the A5 light chain variable region; and vector Db.2256.A5 is a polynucleotide encoding the A5 heavy chain variable region.

In other embodiments, the anti-trkC agonist antibody comprises one or more CDR(s) of antibody A5 (such as one, two, three, four, five, or, in some embodiments, all six CDR(s) from A5). Determination of CDR regions is well within the skill of the art. There are several techniques for determining CDRs: (1) an approach based on cross-species sequence variability (i.e., Kabat et al. Sequences of Proteins of Immunological Interest, (5th ed., 1991, National Institutes of Health, Bethesda Md.)); (2) an approach based on crystallographic studies of antigen-antibody complexes (Al-lazikani et al (1997) J. Molec. Biol. 273:927-948)). Identification of CDRs is well within the skill of the art. In some embodiments, the CDRs comprise the Kabat CDR. In other embodiments, the CDRs are the Chothia CDR. In still other embodiments, the CDR comprises both the Kabat and Chothia CDRs.

Antibodies can encompass monoclonal antibodies, polyclonal antibodies, antibody fragments (e.g., Fab, Fab′, F(ab′)₂, Fv, Fc, etc.), chimeric antibodies, single chain (ScFv), mutants thereof, fusion proteins comprising an antibody portion, and any other modified configuration of the immunoglobulin molecule that comprises an antigen recognition site of the required specificity. The antibodies may be murine, rat, human, or any other origin (including humanized antibodies). Thus, the agonist anti-trkC antibody may be a human antibody (such as antibody 6.4.1 (PCT Publication No. WO 01/98361)) or may be a humanized antibody (including humanized monoclonal antibody A5).

The agonist anti-trkC antibody may bind human trkC. The agonist anti-trkC antibody may also bind human and rodent trkC. In some embodiments, the agonist anti-trkC antibody may bind human and rat trkC. In some embodiments, the anti-trkC antibody may bind human and mouse trkC. In one embodiment, the antibody is an antibody that recognizes one or more epitopes on human trkC extracellular domain. In another embodiment, the antibody is a mouse or rat antibody that recognizes one or more epitopes on human trkC extracellular domain. In some embodiments, the antibody binds human trkC and does not significantly bind trkC from another mammalian species (in some embodiments, vertebrate species). In some embodiments, the antibody binds human trkC as well as one or more trkC from another mammalian species (in some embodiments, vertebrate species). In another embodiment, the antibody recognizes one or more epitopes on a trkC selected from one or more of: primate, canine, feline, equine, and bovine. In some embodiments, the antibody binds trkC and does not significantly cross-react (bind) with other neurotrophin receptors (such as the related neurotrophin receptors, trkA and/or trkB). In some embodiments, the antibody binds trkC, and further binds trkA and/or trkB.

The epitope(s) recognized by the trkC agonist antibody can be continuous or discontinuous. In some embodiments, the antibody binds essentially the same trkC epitope as an antibody selected from any one or more of the following: 6.1.2, 6.4.1, 2345, 2349, 2.5.1, 2344, 2248, 2250, 2253, and 2256. See PCT Publication No. WO 01/98361. Examples of epitopes to which an antibody may be directed include but are not limited to domain V and/or domain IV of trkC. In another embodiment, the epitope includes one or more of the following residues: L284, E287, and N335 of human trkC. See Urfer et al. J. Biol. Chem. (1998) 273:5829-5840). In still other embodiments, the antibody comprises a modified constant region, such as a constant region that is immunologically inert, e.g., does not trigger a complement mediated lysis or does not stimulate antibody-dependent cell mediated cytotoxicity (ADCC). In other embodiments, the constant region is modified as described in Eur. J. Immunol. (1999) 29:2613-2624; PCT Application No. PCT/GB99/01441; and/or UK Patent application No. 9809951.8. In some embodiments, the constant region comprises the human heavy chain IgG2a constant region containing the following mutations: A330P331 to S330S331 (amino acid numbering with reference to the wildtype IgG2a sequence; see Eur. J. Immunol. (1999) 29:2613-2624).

The binding affinity of anti-trkC agonist antibody to trkC may be any of about 500 nM, 400 nM, 300 nM, 200 nM, 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM. In some embodiments, the binding affinity is any of about 100 nM, about 50 nM, about 10 nM, about 1 nM, about 500 pM, about 100 pM, or about 50 pM, or less than about 50 pM. In some embodiments, the binding affinity is less than any of about 100 nM, about 50 nM, about 10 nM, about 1 nM, about 500 pM, about 100 pM, or about 50 pM. In still other embodiments, the binding affinity is about 2 pM, about 5 pM, about 10 pM, about 15 pM, about 20 pM, about 40 pM, or greater than about 40 pM. As is well known in the art, binding affinity can be expressed as K_{D}, or dissociation constant, and an increased binding affinity corresponds to a decreased K_{D}. The binding affinity of mouse anti-trkC agonist monoclonal antibody 2256 to human trkC is about 40 nM, as assessed using BIAcore analysis, and the binding affinity of humanized anti-trkC agonist antibody A5 (described herein) to human trkC is about 0.28 nM, as assessed using BIAcore analysis.

One way of determining binding affinity of antibodies to trkC is by measuring binding affinity of monofunctional Fab fragments of the antibody. To obtain monofunctional Fab fragments, an antibody (for example, IgG) can be cleaved with papain or expressed recombinantly. The affinity of an anti-trkC Fab fragment of an antibody can be determined by surface plasmon resonance (BIAcore3000™ surface plasmon resonance (SPR) system, BIAcore, INC, Piscaway N.J.). CM5 chips can be activated with N-ethyl-N′-(3-dimethylaminopropyl)-carbodiinide hydrochloride (EDC) and N-hydroxysuccinimide (NHS) according to the supplier's instructions. Human trkC-Fc fusion protein (“htrkC”) (or any other trkC, such as rat trkC) can be diluted into 10 mM sodium acetate pH 5.0 and injected over the activated chip at a concentration of 0.0005 mg/mL. Using variable flow time across the individual chip channels, two ranges of antigen density can be achieved: 200-400 response units (RU) for detailed kinetic studies and 500-1000 RU for screening assays. The chip can be blocked with ethanolamine. Regeneration studies have shown that a mixture of Pierce elution buffer (Product No. 21004, Pierce Biotechnology, Rockford, Ill.) and 4 M NaCl (2:1) effectively removes the bound Fab while keeping the activity of htrkC on the chip for over 200 injections. HBS-EP buffer (0.01M HEPES, pH 7.4, 0.15 NaCl, 3 mM EDTA, 0.005% Surfactant P20) is used as running buffer for the BIAcore assays. Serial dilutions (0.1-10× estimated K_{D}) of purified Fab samples are injected for 1 min at 100 μL/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) are obtained simultaneously by fitting the data to a 1:1 Langmuir binding model (Karlsson, R. Roos, H. Fagerstam, L. Petersson, B. (1994). Methods Enzymology 6:99-110) using the BIAevaluation program. Equilibrium dissociation constant (K_{D}) values are calculated as k_{off}/kₒₙ.

In another aspect, antibodies (e.g., human, humanized, mouse, chimeric) that can activate human trkC receptor may be made by using immunogens which express one or more extracellular domains of trkC. One example of an immunogen is cells with high expression of trkC, which can be obtained as described herein. Another example of an immunogen that can be used is a soluble protein (such as a trkC immunoadhesin) which contains the extracellular domain or a portion of the extracellular domain of trkC receptor.

The route and schedule of immunization of the host animal are generally in keeping with established and conventional techniques for antibody stimulation and production, as further described herein. General techniques for production of human and mouse antibodies are known in the art and are described herein.

It is contemplated that any mammalian subject including humans or antibody producing cells therefrom can be manipulated to serve as the basis for production of mammalian, including human, hybridoma cell lines. Typically, the host animal is inoculated intraperitoneally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W. et al., (1982) In Vitro, 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-trkC monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for trkC, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a human or other species of trkC receptor, or a fragment of the human or other species of trkC receptor, or a human or other species of trkC receptor or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaradehyde, succinic anhydride, SOCl2, or R1N═C═NR, where R and R1 are different alkyl groups can yield a population of antibodies (e.g., monoclonal antibodies). Another example of an immunogen is cells with high expression of trkC, which can be obtained from recombinant means, or by isolating or enriching cells from a natural source that express a high level of trkC. These cells may be of human or other animal origin, and may be used as an immunogen as directly isolated, or may be processed in such that immunogenicity is increased, or trkC expression (of a fragment of trkC) is increased or enriched. Such processing includes, but is not limited to, treatment of the cells or fragments thereof with agents designed to increase their stability or immunogenicity, such as, e.g., formaldehyde, glutaraldehyde, ethanol, acetone, and/or various acids. Further, either before or after such treatment the cells may be processed in order to enrich for the desired immunogen, in this case trkC or fragment thereof. These processing steps can include membrane fractionation techniques, which are well known in the art.

If desired, the anti-trkC antibody (monoclonal or polyclonal) of interest may be sequenced and the polynucleotide sequence may then be cloned into a vector for expression or propagation. The sequence encoding the antibody of interest may be maintained in a vector in a host cell and the host cell can then be expanded and frozen for future use. As an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to trkC receptor and greater efficacy in activating trkC receptor. It will be apparent to one of skill in the art that one or more polynucleotide changes can be made to the anti-trkC antibody and still maintain its binding ability to trkC extracellular domain or epitopes of trkC.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640. For example, the constant region may be engineered to more resemble human constant regions to avoid immune response if the antibody is used in clinical trials and treatments in humans. See, for example, U.S. Pat. Nos. 5,997,867 and 5,866,692.

In the recombinant humanized antibodies, the Fcγ portion can be modified to avoid interaction with Fcγ receptor and the complement immune system. This type of modification was designed by Dr. Mike Clark from the Department of Pathology at Cambridge University, and techniques for preparation of such antibodies are described in PCT Publication No. WO 99/58572, published Nov. 18, 1999.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J. Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. The antibody constant region can be engineered such that it is immunologically inert, e.g., does not trigger a complement mediated lysis or does not stimulate antibody-dependent cell mediated cytotoxicity (ADCC). In other embodiments, the constant region is modified as described in Eur. J. Immunol. (1999) 29:2613-2624; PCT Application No. PCT/GB99/01441; and/or UK Patent Application No. 9809951.8. See, e.g. PCT/GB99/01441; UK patent Application No. 9809951.8. Other methods of humanizing antibodies that may also be utilized are disclosed by Daugherty et al., Nucl. Acids Res. 19:2471-2476 (1991) and in U.S. Pat. Nos. 6,180,377; 6,054,297; 5,997,867; 5,866,692; 6,210,671; 6,350,861; and PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

The antibody may be a bispecific antibody, a monoclonal antibody that has binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; and EP 03089). Heteroconjugate antibodies may be made using any convenient cross-linking methods. Suitable cross-linking agents and techniques are well known in the art, and are described in U.S. Pat. No. 4,676,980.

Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco), transgenic milk, or in other organisms. Methods for expressing antibodies recombinantly in plants or milk have been disclosed. See, for example, Peeters et al. (2001) Vaccine 19:2756; Lonberg, N. and D. Huszar (1995) Int. Rev. Immunol 13:65; and Pollock et al. (1999) J. Immunol Methods 231:147. Methods for making derivatives of antibodies, e.g., humanized, single chain, etc. are known in the art.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Single chain Fv fragments may also be produced, such as described in Iliades et al., 1997, FEBS Letters, 409:437-441. Coupling of such single chain fragments using various linkers is described in Kortt et al., 1997, Protein Engineering, 10:423-433. A variety of techniques for the recombinant production and manipulation of antibodies are well known in the art.

Antibodies may be modified as described in PCT Publication No. WO 99/58572, published Nov. 18, 1999. These antibodies comprise, in addition to a binding domain directed at the target molecule, an effector domain having an amino acid sequence substantially homologous to all or part of a constant domain of a human immunoglobulin heavy chain. These antibodies are capable of binding the target molecule without triggering significant complement dependent lysis, or cell-mediated destruction of the target. Preferably, the effector domain is capable of specifically binding FcRn and/or FcγRIIb. These are typically based on chimeric domains derived from two or more human immunoglobulin heavy chain C_{H}2 domains. Antibodies modified in this manner are preferred for use in chronic antibody therapy, to avoid inflammatory and other adverse reactions to conventional antibody therapy.

The antibodies made either by immunization of a host animal or recombinantly should exhibit any one or more of the following characteristics: (a) bind to trkC receptor; (b) bind to one or more epitopes of trkC receptor; (c) bind to trkC receptor and activate trkC biological activity(ies) or one or more downstream pathways mediated by trkC signaling function(s); (d) promote trkC receptor dimerization; (e) increase trkC receptor activation; (f) display favorable pharmacokinetic and bioavailability properties; (g) promote the development, survival, function, maintenance and/or regeneration of cells; (h) bind to trkC receptor and treat, prevent, reverse, or ameliorate one or more symptoms of a lower motor neuron disease.

Immunoassays and flow cytometry sorting techniques such as fluorescence activated cell sorting (FACS) can also be employed to isolate antibodies that are specific for trkC.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

DNA encoding agonist anti-trkC antibodies may be sequenced, as is known in the art. See PCT Publication No. WO 01/98361. Generally, the monoclonal antibody is readily isolated and sequenced using conventional procedures (e.g., by using oligonucleotide probes that are capable of binding specifically to genes encoding the heavy and light chains of the monoclonal antibodies). The hybridoma cells serve as a preferred source of such cDNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81: 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-trkC monoclonal antibody herein. The DNA encoding the agonist anti-trkC antibody (such as an antigen binding fragment thereof) may also be used for delivery and expression of agonist anti-trkC antibody in a desired cell, as described here. DNA delivery techniques are further described herein.

Anti-trkC antibodies may be characterized using methods well-known in the art. For example, one method is to identify the epitope to which it binds, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-trkC antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-trkC antibody. In another example, the epitope to which the anti-trkC antibody binds can be determined in a systematic screening by using overlapping peptides derived from the trkC extracellular sequence and determining binding by the anti-trkC antibody. According to the gene fragment expression assays, the open reading frame encoding trkC is fragmented either randomly or by specific genetic constructions and the reactivity of the expressed fragments of trkC with the antibody to be tested is determined. The gene fragments may, for example, be produced by PCR and then transcribed and translated into protein in vitro, in the presence of radioactive amino acids. The binding of the antibody to the radioactively labeled trkC fragments is then determined by immunoprecipitation and gel electrophoresis. Certain epitopes can also be identified by using large libraries of random peptide sequences displayed on the surface of phage particles (phage libraries).

Yet another method which can be used to characterize an anti-trkC antibody is to use competition assays with other antibodies known to bind to the same antigen, i.e., trkC extracellular domain to determine if the anti-trkC antibody binds to the same epitope as other antibodies. Competition assays are well known to those of skill in the art. Examples of antibodies useful in competition assays include the following: antibodies 6.1.2, 6.4.1, 2345, 2349, 2.5.1, 2344, 2248, 2250, 2253, and 2256. See PCT Publication No. WO 01/98361.

Epitope mapping can also be performed using domain swap mutants as described in PCT Publication No. WO 01/98361. Generally, this approach is useful for anti-trkC antibodies that do not significantly cross-react with trkA or trkB. Domain-swap mutants of trkC can be made by replacing extracellular domains of trkC with the corresponding domains from trkB or trkA. The binding of each agonist anti-trkC antibody to various domain-swap mutants can be evaluated and compared to its binding to wild type (native) trkC using ELISA or other method known in the art. In another approach, alanine scanning can be performed. Individual residues of the antigen, the trkC receptor, are systematically mutated to another amino acid (usually alanine) and the effect of the changes is assessed by testing the ability of the modified trkC to bind to antibody using ELISA or other methods known in the art.

Identification of Agonist Anti-trkC Antibodies

Agonist antibodies may be identified using art-recognized methods, including one or more of the following methods. For example, the kinase receptor activation (KIRA) assay described in U.S. Pat. Nos. 5,766,863 and 5,891,650 may be used. This ELISA-type assay is suitable for qualitative or quantitative measurement of kinase activation by measuring the autophosphorylation of the kinase domain of a receptor protein tyrosine kinase (rPTK, e.g. trk receptor), as well as for identification and characterization of potential agonist or antagonists of a selected rPTK. The first stage of the assay involves phosphorylation of the kinase domain of a kinase receptor, in the present case a trkC receptor, wherein the receptor is present in the cell membrane of a eukaryotic cell. The receptor may be an endogenous receptor or nucleic acid encoding the receptor, or a receptor construct, may be transformed into the cell. Typically, a first solid phase (e.g., a well of a first assay plate) is coated with a substantially homogeneous population of such cells (usually a mammalian cell line) so that the cells adhere to the solid phase. Often, the cells are adherent and thereby adhere naturally to the first solid phase. If a “receptor construct” is used, it usually comprises a fusion of a kinase receptor and a flag polypeptide. The flag polypeptide is recognized by the capture agent, often a capture antibody, in the ELISA part of the assay. An analyte, such as a candidate agonist, is then added to the wells having the adherent cells, such that the tyrosine kinase receptor (e.g. trkC receptor) is exposed to (or contacted with) the analyte. This assay enables identification of agonist ligands for the tyrosine kinase receptor of interest (e.g. trkC). Following exposure to the analyte, the adhering calls are solubilized using a lysis buffer (which has a solubilizing detergent therein) and gentle agitation, thereby releasing cell lysate which can be subjected to the ELISA part of the assay directly, without the need for concentration or clarification of the cell lysate.

The cell lysate thus prepared is then ready to be subjected to the ELISA stage of the assay. As a first step in the ELISA stage, a second solid phase (usually a well of an ELISA microciter plate) is coated with a capture agent (often a capture antibody) that binds specifically to the tyrosine kinase receptor, or, in the case of a receptor construct, to the flag polypeptide. Coating of the second solid phase is carried out so that the capture agent adheres to the second solid phase. The capture agent is generally a monoclonal antibody, but, as is described in the examples herein, polyclonal antibodies or other agents may also be used. The cell lysate obtained is then exposed to, or contacted with, the adhering capture agent so that the receptor or receptor construct adheres to (or is captured in) the second solid phase. A washing step is then carried out, so as to remove unbound cell lysate, leaving the captured receptor or receptor construct. The adhering or captured receptor or receptor construct is then exposed to, or contacted with, an anti-phosphotyrosine antibody which identifies phosphorylated tyrosine residues in the tyrosine kinase receptor. In the preferred embodiment, the anti-phosphotyrosine antibody is conjugated (directly or indirectly) to an enzyme which catalyses a color change of a non-radioactive color reagent. Accordingly, phosphorylation of the receptor can be measured by a subsequent color change of the reagent. The enzyme can be bound to the anti-phosphotyrosine antibody directly, or a conjugating molecule (e.g., biotin) can be conjugated to the anti-phosphotyrosine antibody and the enzyme can be subsequently bound to the anti-phosphotyrosine antibody via the conjugating molecule. Finally, binding of the anti-phosphotyrosine antibody to the captured receptor or receptor construct is measured, e.g., by a color change in the color reagent.

Following initial identification, the agonist activity of a candidate antibody can be further confirmed and refined by bioassays, known to test the targeted biological activities. For example, the ability of anti-trkC monoclonal antibodies to agonize trkC can be tested in the PC12 neurite outgrowth assay using PC12 cells transfected with full-length human trkC (Urfer et al., Biochem. 36: 4775-4781 (1997); Tsoulfas et al., Neuron 10: 975-990 (1993)). This assay measures the outgrowth of neurite processes by rat pheocytochroma cells (PC12) in response to stimulation by appropriate ligands. These cells express endogenous trkA and are therefore responsive to NGF. However, they do not express endogenous trkC and are therefore transfected with trkC expression construct in order to elicit response to trkC agonists. After incubating the transfected cells with anti-trkC antibodies, neurite outgrowth is measured, and e.g., cells with neurites exceeding 2 times the diameter of the cell are counted. Anti-trkC antibodies that stimulate neurite outgrowth in transfected PC12 cells demonstrate trkC agonist activity.

The activation of trkC may also be determined by using various specific neurons at specific stages of embryonic development. Appropriately selected neurons can be dependent on trkC activation for survival, and so it is possible to determine the activation of trkC by following the survival of these neurons in vitro. Addition of candidate antibodies to primary cultures of appropriate neurons will lead to survival of these neurons for a period of at least several days if the candidate antibodies activate trkC. This allows the determination of the ability of the candidate antibody to activate trkC. In one example of this type of assay, the trigeminal ganglion from an E11 mouse embryo is dissected, dissociated and the resultant neurons are plated in a tissue culture dish at low density. The candidate antibodies are then added to the media and the plates incubated for 24-48 hours. After this time, survival of the neurons is assessed by any of a variety of methods. Samples which received an agonist anti-trkC antibody will typically display an increased survival rate over samples which receive a control antibody, and this allows the determination of the presence of an agonist anti-trkC antibody. See, e.g., Buchman et al (1993) Development 118(3):989-1001.

Agonist antibodies may be identified by their ability to activate downstream signaling in a variety of cell types that express trkC, either naturally or after transfection of DNA encoding trkC. This trkC may be human or other mammalian (such a rodent or primate) trkC. The downstream signaling cascade may be detected by changes to a variety of biochemical or physiological parameters of the trkC expressing cell, such as the level of protein expression or of protein phosphorylation of proteins or changes to the metabolic or growth state of the cell (including neuronal survival and/or neurite outgrowth, as described herein). Methods of detecting relevant biochemical or physiological parameters are known in the art.

Administration of Agonist Anti-trkC Antibodies

Various formulations of agonist anti-trkC antibodies may be used for administration. In some embodiments, an agonist anti-trkC antibody may be administered neat. In some embodiments, an agonist anti-trkC antibody is administered in a composition comprising a pharmaceutically acceptable excipient. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Agonist anti-trkC antibodies can be formulated for administration by injection (e.g., intraperitoneally, intravenously, subcutaneously, intramuscularly, etc.). Accordingly, the antibodies may be combined with pharmaceutically acceptable vehicles such as saline, Ringer's solution, dextrose solution, and the like. The particular dosage regimen, i.e., dose, timing and repetition, will depend on the particular individual and that individual's medical history. Generally, a dose of less than about 1 ug/kg body weigh, at least about 1 ug/kg body weight; at least about 2 ug/kg body weight, at least about 5 ug/kg body weight, at least about 10 ug/kg body weight, at least about 20 ug/kg body weight, at least about 50 ug/kg body weight, at least about 100 ug/kg body weight, at least about 200 ug/kg body weight, at least about 500 ug/kg body weight, at least about 1 mg/kg, body weight, at least about 2 mg/kg body weight, at least about 5 mg /kg body weight, at least about 10 mg/kg body weight, at least about 30 mg/kg body weight, or more (such as about 50 mg/kg, about 100 mg/kg, about 200 mg/kg or about 500 mg/kg) is administered.

Empirical considerations, such as the half-life, generally will contribute to the determination of the dosage. Antibodies which are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on maintaining an effective concentration of agonist anti-trkC antibody in the patient and suppression and/or amelioration and/or delay of one or more symptoms of a lower motor neuron disease. Alternatively, sustained continuous release formulations of agonist anti-trkC antibodies may be appropriate. Various formulations and devices for achieving sustained release are known in the art. Administration of an agonist anti-trkC antibody in accordance with the method in the present invention can be continuous or intermittent, depending, for example, upon the recipient's physiological condition, whether the purpose of the administration is therapeutic or prophylactic, and other factors known to skilled practitioners. The administration of an agonist anti-trkC antibody may be essentially continuous over a preselected period of time or may be in a series of spaced dose, e.g., either before; during; or after developing symptoms of a lower motor neuron disease; before, and during; before and after; during and after; and/or before, during, and after developing symptoms of a lower motor neuron disease.

Generally, for administration of agonist anti-trkC antibodies, an initial candidate dosage can be about 2 mg/kg. For the purpose of the present invention, a typical daily dosage might range from about 30 μg/kg to 100 mg/kg or more, depending on the factors mentioned above. For repeated administrations over several days or longer, depending on the condition, the treatment is sustained until a desired suppression of disease symptoms occurs or until sufficient therapeutic levels are achieved to treat or prevent a lower motor neuron disease. An exemplary dosing regimen comprises administering an initial dose of about 2 mg/kg, followed by a weekly maintenance dose of about 1 mg/kg of the trkC agonist antibody, or followed by a maintenance dose of about 1 mg/kg every other week.

In one embodiment, dosages for an antibody may be determined empirically in individuals who have been given one or more administration(s) of an agonist anti-trkC antibody that activates trkC receptor to treat a lower motor neuron disease. Individuals are given incremental dosages of an agonist anti-trkC antibody. To assess efficacy of agonist anti-trkC antibodies, an indicator of a lower motor neuron disease state can be followed as described herein.

Other formulations include suitable delivery forms known in the art including, but not limited to, carriers such as liposome. See, for example, Mahato et al. (1997) Pharm. Res. 14:853-859. Liposomal preparations include, but are not limited to, cytofectins, multilamellar vesicles and unilamellar vesicles.

The formulations to be used for in vivo administration must be sterile. This is readily accomplished by, for example, filtration through sterile filtration membranes. Therapeutic agonist anti-trkC antibody compositions are generally placed into a container having a sterile access port, for example, an intravenous solution bag or vial having a stopper pierceable by a hypodermic injection needle.

The agonist anti-trkC antibody is administered to a individual in accord with known methods, such as intravenous administration, e.g., as a bolus or by continuous infusion over a period of time, by intramuscular, intraperitoneal, subcutaneous, oral, intrathecal, or topical routes. Agonist anti-trkC antibody can also be administered by inhalation. Commercially available nebulizers for liquid formulations, including jet nebulizers and ultrasonic nebulizers are useful for administration. Liquid formulations can be directly nebulized and lyophilized powder can be nebulized after reconstitution. Alternatively, agonist anti-trkC antibody can be aerosolized using a fluorocarbon formulation and a metered dose inhaler, or inhaled as a lyophilized and milled powder.

In some embodiments, more than one antibody may be present. The antibodies can be the same or different from each other. In some embodiments, at least one, at least two, at least three, at least four, at least five different trkC agonist antibodies are present. Preferably those antibodies have complementary activities that do not adversely affect each other.

A polynucleotide encoding an agonist anti-trkC antibody (such as an antigen binding fragment thereof) may also be used for delivery and expression of agonist anti-trkC antibody in a desired cell. It is apparent that an expression vector can be used to direct expression of an agonist anti-trkC antibody. The expression vector can be administered intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding an agonist anti-trkC antibody can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5, 219,740; 4,777,127; GB Patent No. 2,200,651; and EP 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0 524 968. Additional approaches are described in Philip, Mol. Cell Biol. (1994) 14:2411, and in Woffendin, Proc. Natl. Acad. Sci. (1994) 91:1581.

The agonist anti-trkC antibody may be administered in conjunction with one or more other agents (such as one or more neurotrophins) for treating lower motor neuron diseases, i.e., administered in combination with, in concert with, or sequentially with the one or more agents. For example, an agonist anti-trkC antibody may be administered in conjunction with NT-4. Administration in conjunction, as used herein, comprises simultaneous administration and/or administration at different times. Administration in conjunction also encompasses administration as a co-formulation (i.e., the agonist anti-trkC antibody and the other agent are present (combined) in the same composition) and/or administration as separate compositions. As used herein, “administration in conjunction” is meant to encompass any circumstance wherein agonist anti-trkC antibody and the other agent(s) are administered in an effective amount to an individual. As further discussed herein, it is understood that the agonist anti-trkC antibody and the other agent(s) can be administered at different dosing frequencies and/or intervals. For example, an agonist anti-trkC antibody may be administered weekly, while other agent(s) (e.g., NT-4) may be administered more frequently. It is understood that an agonist anti-trkC antibody and the other agent(s) can be administered using the same route of administration or different routes of administration, and that different dosing regimens may change over the course of administration(s). Administration may be before the onset of a lower motor neuron disease.

For administration of an agonist anti-trkC antibody in conjunction with a neurotrophin, a polynucleotide encoding the neurotrophin (e.g., CNTF, NT-4) may also be used for delivery and expression of the neurotrophin in a desired cell (e.g., skeletal muscle cells) utilizing an expression vector described herein.

Methods of Assessing Efficacy of Treatment with Agonist Anti-trkC Antibodies

Assessment of treatment efficacy can be performed on several different levels. Assessment may be made by monitoring clinical signs (e.g., strength tests, electrophysiological responses, or molecular changes). These may include parameters as determined by a standard neurological examination, or patient interview or may be determined by more specialized quantitative testing, e.g., as described herein. These more specialized quantitative tests may include, but are not limited to, determination of conduction velocity of the affected neurons by means such as microneurography, electromyography (EMG), voluntary muscle power measurements such as grip strength, syllable repetition, walking speed measured by the time taken to walk 15 feet (4.57 meters), respiratory function tests including measurement of the forced vital capacity (FVC), the occurrence of selected events associated with progression of respiratory disability, hearing, tests of balance, specialized tests of proprioception, or kinesthetic sense, tests of strength, electromyography, tests of autonomic function, including, but not limited to, test of blood pressure control, tests of heart rate response to various physiological and pharmacological stimuli. These tests may also include tests of motor skill or strength.

Compositions for Use in Treatment of Lower Motor Neuron Diseases

The invention also provides compositions for use in any of the methods described herein. The compositions used in the methods of the invention comprise an effective amount of an agonist anti-trkC antibody. Examples of such compositions, as well as how to formulate, are also described in an earlier section and below. The invention also provides any of the compositions described for any use described herein whether in the context of use as medicament and/or use for manufacture of a medicament.

The composition used in the present invention can further comprise pharmaceutically acceptable carriers, excipients, or stabilizers (Remington: The Science and practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In one aspect, the invention provides compositions comprising an agonist anti-trkC antibody. In other embodiments, the agonist anti-trkC antibody recognizes human trkC. In still other embodiments, the agonist anti-trkC antibody is humanized (such as antibody A5 described herein). In other embodiments, the anti-trkC agonist antibody comprises one or more CDR(s) of antibody A5 (such as one, two three, four, five or, in some embodiments, all six CDRs from A5). In still other embodiments, the anti-trkC agonist antibody comprises the amino acid sequence of the heavy chain variable region shown in Table 1 (SEQ ID NO:1) and the amino acid sequence of the light chain variable region shown in Table 2 (SEQ ID NO:2). In still other embodiments, the agonist anti-trkC antibody is a human antibody.

It is understood that the compositions can comprise more than one agonist anti-trkC antibody (e.g., a mixture of agonist anti-trkC antibodies that recognize different epitopes of trkC). Other exemplary compositions comprise more than one agonist anti-trkC antibody that recognize the same epitope(s), or different species of agonist anti-trkC antibodies that bind to different epitopes of trkC.

The agonist anti-trkC antibody and compositions thereof can also be used in conjunction with other agents that serve to enhance and/or complement the effectiveness of the agonist anti-trkC antibody. For example, such additional compounds may include compounds known to be useful for the treatment of lower motor neuron diseases, one or more neurotrophins (including CNTF, NT-3, NT-4, BDNF, and GDNF), and agonist anti-trkB agonists. Such molecules are suitably present in combination in amounts that are effective for the purpose intended. The trkC agonist antibody and compositions thereof can also be used in conjunction with other agents that serve to enhance and/or complement the effectiveness of the antibodies.

Kits

The invention also provides kits for use in the instant methods. Kits of the invention include one or more containers comprising an anti-trkC agonist antibody and, in some embodiments, further comprise instructions for use in accordance with any of the methods of the invention described herein (such as methods for treating a lower motor neuron disease). In some embodiments, these instructions comprise a description of selecting an individual suitable for treatment based on identifying whether that individual has a lower motor neuron disease and/or is at risk of developing the lower motor neuron disease, and may further describe administration of the trkC agonist antibody for treatment and/or prevention of the disease. The invention also provides any of the kits described for any use described herein whether in the context of use as medicament and/or use for manufacture of a medicament.

Thus, in one embodiment, the invention provides kits comprising an agonist anti-trkC antibody. In some embodiments, the invention provides kits for use with the methods described herein comprising an agonist anti-trkC antibody. The kits of this invention are in suitable packaging. Suitable packaging includes, but is not limited to, vials, bottles, jars, flexible packaging (e.g., sealed Mylar or plastic bags), and the like. In some embodiments, the kit comprises a container and a label or package insert(s) on or associated with the container. The label or package insert indicates that the composition is useful for treating, preventing or ameliorating a lower motor neuron disease. Instructions may be provided for practicing any of the methods described herein. The container holds a composition which is effective for treating a lower motor neuron disease, and may have a sterile access port (for example the container may be an intravenous solution bag or a vial having a stopper pierceable by a hypodermic injection needle). At least one active agent in the composition is a trkC agonist antibody. The container may further comprise a second pharmaceutically active agent. Kits may optionally provide additional components such as buffers and interpretive information.

The following Examples are provided to illustrate but not limit the invention.

### EXAMPLES

### Effects of an Agonist Anti-trkC Antibody and NT-4 on SMARD1 Animal Model

Spinal muscular atrophy with respiratory distress type 1 (SMARD1) is a fatal autosomal recessive disorder of infants. It is characterized by lower motor neuron degeneration, progressive muscle paralysis and respiratory failure, for which no effective treatment exists. The phenotype of nmd (neuromuscular degeneration) mice closely resembles the human SMARD1. The identification of the mutated mouse gene in nmd mice, Ighmbp2, led to the discovery of mutations of the homologous gene in humans with SMARD1. We have studied the nmd mouse model with in vivo electrophysiological techniques and evaluated the efficacy of Mab2256, a monoclonal antibody with agonist effect on the tyrosine kinase receptor C, trkC, on disease progression in nmd mice. Treatment with Mab2256 resulted in a significant but transient improvement of muscle strength in nmd mice, as well as normalization of the neuromuscular depression during high frequency nerve stimulation. These results suggest the potential of using monoclonal agonist antibodies for neurotrophin receptors in lower motor neuron diseases such as SMARD1.

In the present study we performed an in vivo electrophysiological characterization of hind limb and diaphragm muscles of the nmd mouse model of SMARD1 and we evaluated the potential efficacy of an agonistic monoclonal antibody (Mab2256) for trkC receptor in this mouse model. We show that Mab2256 treatment prevented the initial decline of muscular strength and it led to the electrophysiological improvement of muscular function, consisting of restoration to normality of the level of depression during repetitive electrical stimulation. However, such initial improvements did not translate into muscle fiber preservation or survival benefit, highlighting the areas for further optimization of this therapeutic strategy.

Results

Agonistic and Pharmacokinetic Properties of the trkC Antibody Mab2256

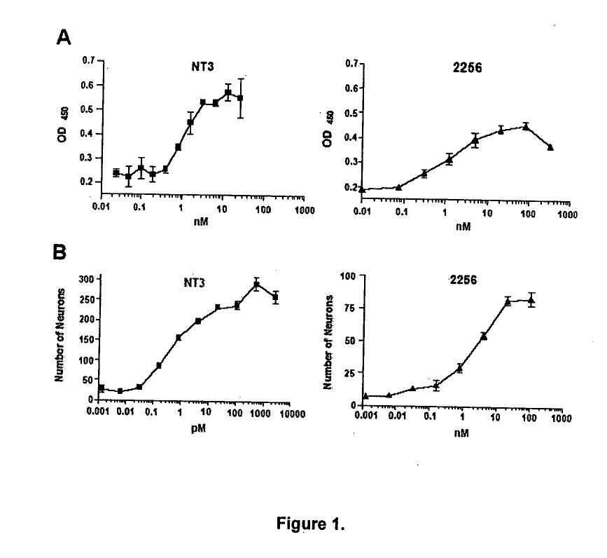

Monoclonal antibodies against the human trkC extracellular domain were generated and screened for agonist activity using a cell based receptor tyrosine phosphorylation assay (16). One monoclonal antibody, Mab2256, of the murine IgG₁ isotype was found to be a specific binder of trkC receptor without cross reactivity with trkA or trkB (data not shown). In the stable trkC expressing CHO cells, Mab2256 induces trkC tyrosine phosphorylation with the half maximum effective concentration (EC₅₀) of 0.87 nM, whereas NT3 does so with EC₅₀ of 1.09 nM (FIG. 1A). Consistent with its ability to activate trkC receptor, Mab2256 also supports the survival of the embryonic rat trigeminal neuron cultures in a dose dependent fashion with an EC₅₀ of 2.58 nM, while NT3 has an EC₅₀ of 0.73 pM (FIG. 1B).

Next we investigated the pharmacokinetic property of Mab2256. We found the elimination phase half life (t_{1/2}) of Mab2256 from serum to be ˜199 hours when given intraperitoneally in mice. This is the long circulating half life expected of an antibody, and it compares favorably with the published plasma half life of the endogenous trkC agonist, NT3, at around 1.28 minutes (17).

Effect of Mab2256 on Motor Performance and Survival in nmd Mice

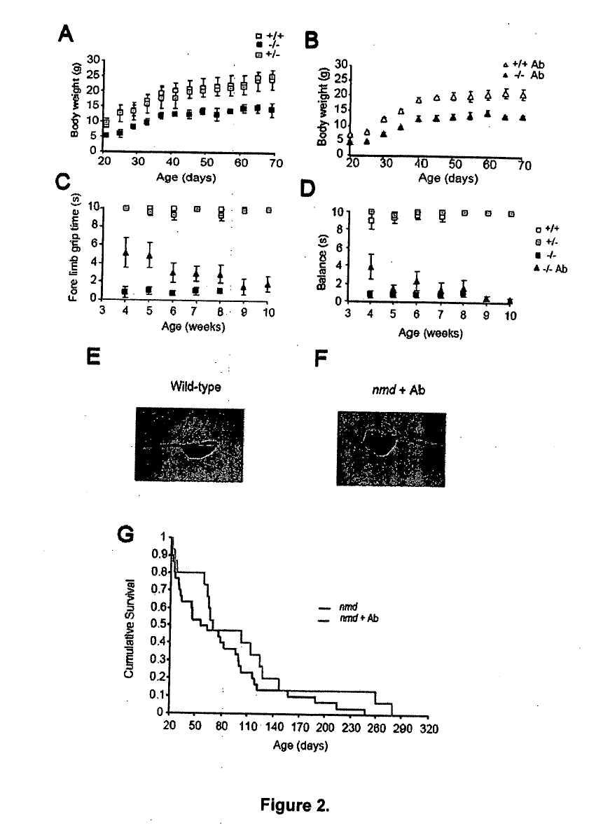

Mutant mice appeared indistinguishable from wild-type at birth but they fed and grew poorly after 2 weeks of birth. Hence the homozygous mutants were easily identifiable from wild-type and heterozygous littermates by their lower body weight (FIG. 2A). In addition, mutant mice exhibited progressive loss of muscle mass and a marked decrease in fore- and hind-limb grip strength (8-11). They could not support their body against gravity, were unable to grasp a cage cover and gradually lost muscle mass of the shoulder and pelvic girdles.

We examined whether treatment with Mab2256 (5 mg/kg body weight, 2 times per week, starting on postnatal day 20, i.e. P20) could prevent the progressive loss of muscular function that occurs in nmd mice. The presence of the monoclonal antibody in serum was confirmed by ELISA analysis in mice injected with Mab2256 (n=10) or PBS (n=5) for 6-8.5 weeks. The mean concentration of the Mab2256 antibody in serum was 10.6±3.4 μg/mL (n=10 of Mab2256 injected mice). No side effects were observed in wild-type and nmd Mab2256-treated mice. There was no significant differences in body weight between treated and untreated mice (FIG. 2A, B): on P60 the body weight of Mab2256-treated mutants was 14.5±0.2 g (n=16), and that of untreated or vehicle control 14.2±0.83 g (n=8).

Standard neurological examinations were performed in untreated and Mab2256-treated mice from 4-10 weeks of age. To explore forelimb gripping strength, mice were suspended upon a metal wire 10 cm above the floor by their forelimbs. The duration that the mouse remained suspended was recorded. Untreated nmd mice (PBS-injected n=5; not-injected n=9) showed a nearly complete loss of grip strength in their forelimbs throughout the entire study (FIG. 2C, filled squares). Strikingly, the Mab2256-treated nmd mice retained the grip strength for several weeks (FIG. 2C, triangles). The mean duration that PBS-injected nmd mice were able to maintain themselves on the wire was 0.8±0.6 sec at four weeks of age (n=5), while at the same age in Mab2256-treated nmd mice it was 5.1±1.6 sec (9 mice, 6 litters) (p<0.03), and 4.9±1.4 sec a week later (p<0.034). Heterozygous performance (FIG. 2C, grey squares; n=8) was identical to that of wild-type siblings (FIG. 2C, white squares).

Motor coordination was also measured by the ability of the nmd mice to maintain themselves on a constant speed rotating rod (test time duration: 10 sec). Untreated nmd mice showed very poor balance in comparison with wild-type and heterozygous littermates (FIG. 2D, E), (p<0.001) while Mab2256-treated nmd mice showed significantly better motor coordination one week after treatment (FIG. 2D, F), (p<0.018). These results indicated that Mab2256 treatment ameliorated disease progression in nmd mice.

To answer whether Mab2256 affected survival, life spans of untreated and Mab2256-treated nmd mice were recorded from weaning to adulthood. No significant increase in life span was observed (Mann-Whitney Rank-Sum test): the median life span of Mab2256-treated mutant mice was 69 days (n=15) while that of untreated nmd mice was 62 days (n=30). (FIG. 2G).

Electrical Neuromuscular Activity in nmd Mice

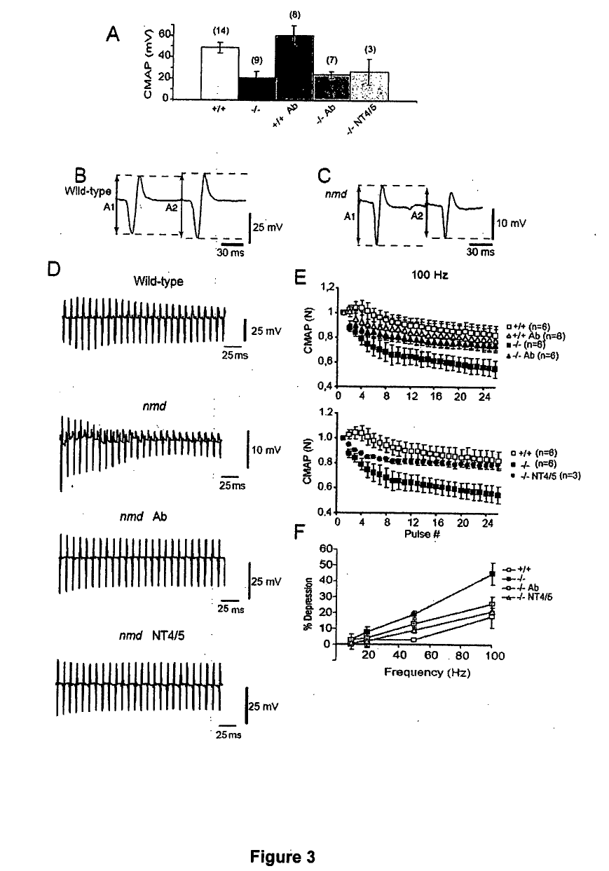

In vivo EMG measurements were performed on P70 in the medial gastrocnemius (MG). We first stimulated the sciatic nerve by a single current pulse of supramaximal amplitude and recorded the compound muscular action potential (CMAP). The mean amplitude of the CMAP in the nmd mouse was reduced to <50% of the control value (FIG. 3A; 22.9±5.6 mV, n=9 and 48.81±4.8 mV, n=14, respectively; p<0.002). The administration of Mab2256 had no significant effect on the mean amplitude of the CMAP in nmd (24±3.1 mV, n=7) or wild-type mice on P70 (61±9.2 mV, n=8) (FIG. 3A), suggesting that the treatment protocol with Mab2256 was not able to stop the loss of motor fibres. To ask if a trkB agonist may be better than a trkC agonist, we also treated a group of mutant mice with the trkB agonist NT-4/5 (5 mg/kg body weight, 2 times per week, starting at P20). In this case the mean amplitude of the CMAP was not altered by NT-4/5 treatment, either (26.7±7.1 mV, n=3 on P70).

Mab2256 and NT-4/5 Restore Normal Levels of High Frequency-Induced Neuromuscular Depression

To further investigate the neuromuscular electrical properties in nmd mice and the effect of Mab2256 and NT-4/5, we used paired-pulses, and short-train stimuli at different frequencies, and studied the electromyographic responses. In the MG, the amplitudes of the CMAP responses (A1 & A2) to paired-pulse supramaximal stimuli (10 ms interval) in nmd mice were much different from those in the wild type. In wild-type mice, the amplitude of the second CMAP (A2) was slightly greater than or equal to the first response (A1) (FIG. 3B). In mutant mice, the amplitude of the second response was 21.4±0.03% smaller than that of the first response (n=7) (FIG. 3C) (p<0.005). The areas under the CMAP curves changed accordingly with their amplitudes, indicating a real change in the number of fibres activated and not a pseudodepression (data not shown).

With 250 ms trains of stimuli at 100 Hz, the amplitude of the CMAPs in untreated wild-type MG showed a consistent pattern: they increased gradually during the first 3-4 stimuli, and then progressively decreased over the stimulation period until reaching a quasi-steady-state value of depression (FIG. 3D, upper trace; FIG. 3E, open squares). This pattern was very different from the one seen in untreated nmd mice that was characterized by a maximal drop in amplitude between the first and the second CMAP of the train, followed by a further decline over the recording period (FIG. 3D, second trace; FIG. 3E, filled squares). In untreated mutants, the depression was fast and the normalized amplitude of the CMAP at the end of the train was much less than in the wild-type (55±6.9%, n=6 and 82.5±7.4%, n=6, respectively) (p<0.02), suggesting an increase in the number of fibres where transmission had fallen bellow threshold for action potential generation. Repetition of the same pattern of stimuli after an interval of no stimulation (2-4 min.) gave a nearly identical pattern of responses to each presentation of the stimuli train.

To asses the efficacy of Mab2256 on neuromuscular function we recorded animals treated with the monoclonal antibody and compared the results with untreated mice. With repetitive nerve stimulation (100 Hz), the normalized amplitude of the CMAP at the quasi-steady-state was significantly larger in Mab2256-treated nmd mice (74±4.1%, n=6) (FIG. 3D, third trace) than in PBS-injected mutants (55±6.9%, n=6) (FIG. 3E, upper graph) (p<0.05). However, the amount of depression of the response at the end of the train was not significantly different in Mab2256-treated and untreated wild-type mice (78±9%, n=8 and 82.5±7.4%, n=6, respectively) (FIG. 3E, upper graph) (p<0.73), suggesting that the treatment with the monoclonal antibody did not alter normal neuromuscular function.

In NT-4/5-treated nmd mice, the normalized amplitude of the final steady state CMAP was also significantly larger at 100 Hz (79±2.5%; n=3) (FIG. 3D, lower trace; FIG. 3E, filled circles in lower graph) than in PBS-injected mutants (p<0.02). The amount of depression at different frequencies (10, 20, 50 and 100 Hz) in wild-type, Mab2256-treated, and untreated mutants are shown in FIG. 3F. These results suggest that both Mab2256 and NT-4/5 were able to restore, almost completely, the normal levels of high frequency-induced neuromuscular depression.

Functional State of Spindle Afferent Fibres in nmd Mice

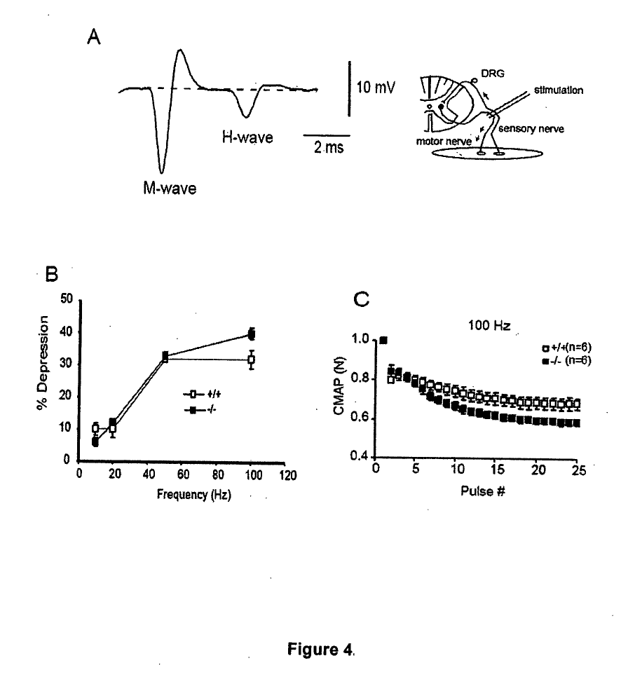

To asses the functional state of muscle spindle afferent fibres in untreated and Mab2256-treated nmd mice, we recorded from the dorsal foot muscles, where the H-waves are easily detectable. H-waves are elicited by the activation of motor fibres through the monosynaptic proprioceptive sensory afferent circuit (inset FIG. 4A) and are preceded by M-waves, which are elicited by the direct stimulation of nerve motor fibres (FIG. 4A). The M/H-wave ratios were not different in wild-type (6.1±1.3, n=6), untreated nmd (6.1±0.9, n=5) and Mab2256-treated nmd mice (7.6±2.6, n=4), suggesting that muscle spindle fibres responsible for the stretch reflex in the dorsal foot muscles are not preferentially lost in mutants and that the treatment did not affect this circuit.

In dorsal foot muscles, we also studied the amount of depression of the EMG responses at different stimulation frequencies in nmd and control mice. There were no significant differences in mean quasi-steady-state depression of the CMAPs between wild type and nmd mice at frequencies from 10 to 50 Hz (FIG. 4B). Only, at 100 Hz (FIG. 4C) the amount of depression was slightly larger in nmd mice (40±1.6%, n=6) than in wild-type (32±2.85%, n=6) (P<0.036) suggesting that the foot dorsal muscles are less affected than the MG in this animal model.

MUNE Analysis

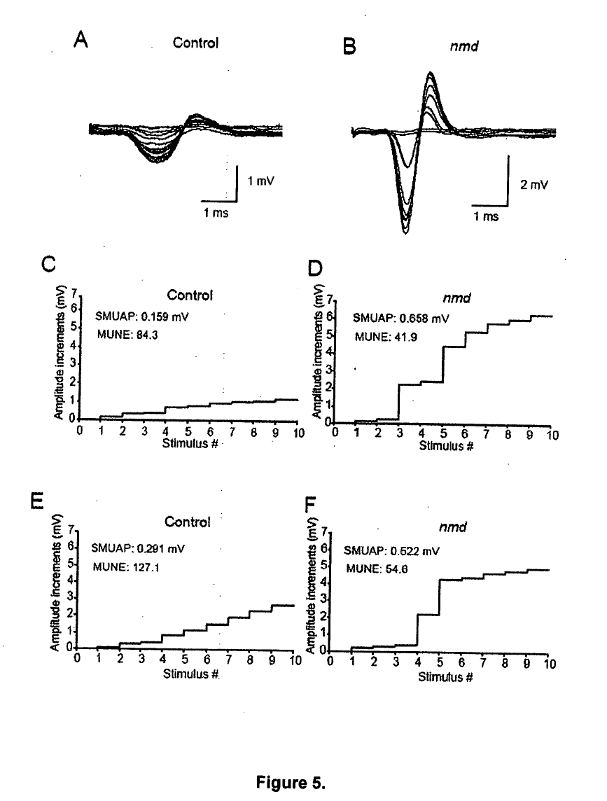

The reduction in the amplitude of the CMAPs recorded in the MG of nmd mice suggests a decrease in the number of functional motor units. We used motor unit number estimation (MUNE) to evaluate the degree of motor neuron loss in the MG of nmd mice. The number of functional motor units was determined at a late period in life of the mutant mice (P215-P230), and compared with that of control littermates. At this age, muscular atrophy of the hindlimbs was very severe in the mutants while no signs of muscle wasting or strength decrement were observed in the heterozygous. Successive incremental stimuli produced regular increments in motor unit potentials in the controls (FIG. 5A) while they elicited abnormally large motor units potentials in nmd mice (FIG. 5B). The final size of the potential after ten “successful” stimuli of increasing strength (i.e. stimuli that elicited an increment in the amplitude of the response) was much larger in the mutants than in the control sib mice due to the presence of large step increments in the mutants (i.e., giant motor units). Quantification of the size of the potentials is shown in the graphs in FIG. 5C-F from two control (heterozygous) and two mutant littermates. The average single motor unit action potential (SMUAP) amplitude was 1.21±0.62 mV (n=3) in nmd mice and 0.178±0.06 mV (n=3) in control mice. Consequently, the MUNE was reduced over 50% in nmd mice (mean value of 37.5±11.4, n=3), as compared with the heterozygous littermates (105.5±12.4, n=3, P<0.0015). Moreover, low intensity stimuli adequate for control mouse failed to elicit any response in the mutants, indicating that the threshold for fibre activation was increased in most of the remaining motor units of the mutant (data not shown). The existence of giant motor units is an indication of axonal sprouting and reinnervation of denervated muscle fibres that probably compensate at least partially for the severe loss in neuromuscular transmission. However, if the mutant strength depended primarily on the giant motor units, the loss of these units may lead to an abrupt failure in muscular function later on.

Diaphragmatic Function in nmd Mice

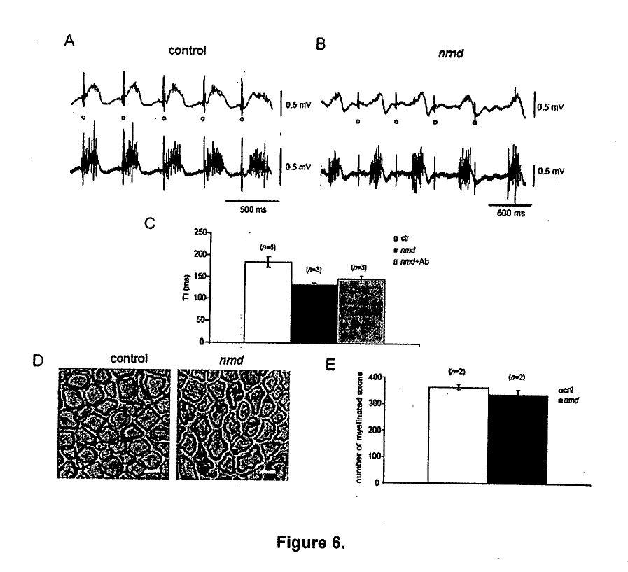

Respiratory failure is a characteristic of human SMARD1 as early as the first year of life (4). To assess the respiratory function in nmd mice and the potential efficacy of the monoclonal antibody, we examine the electrophysiological property of the diaphragm of mice under anaesthetic conditions. Twelve mice older than ten weeks were included in this study: controls (wild-types and heterozygous, n=6), untreated nmd mice (n=3) and Mab2256-treated nmd mice (n=3). The electrical activity in the diaphragm is characterized by alternating bursts of spontaneous action potentials (inspiratory burst) and silent periods that coincide with expiration. Representative recordings from the diaphragm in a control and in an untreated-nmd mouse are shown in the lower traces in FIG. 6A, B. In general, no postinspiratory electrical activity was observed in control and nmd mice. We found that there was little variability in the duration and in the activity of the inspiratory burst (T_{I}). The respiratory frequency in control mice (140.2±15.7 bpm, n=6) was similar to untreated-nmd (141.7±6.2 bpm, n=3) and Mab2256-treated nmd mice (132.9±22.6, n=3). However, there was a significant reduction (28%) in the mean duration of the inspiration (TI) in untreated-nmd mice (131.6±4.1 ms) in comparison with control littermates (184.1±11.7 ms) (P<0.005), suggesting that nmd mice, at late stages of life, have a mild abnormal inspiratory motor discharge. In Mab2256-treated nmd mice TI was 146.3±8.2 ms, slightly larger than in untreated nmd mice. This difference did not reach statistical significance probably owning to the small sample size (P<0.08) (FIG. 6C). Histological examination of phrenic nerves' transverse sections (FIG. 6D) in very old mutants (38 weeks) showed no significant differences in the number of myelinated axons between nmd (337±18 axons, n=2) and control littermates (364±11 axons, n=2) (FIG. 6E). This suggests a functional deficit, rather than an anatomical loss, of the phrenic nerves is underlying the respiratory dysfunction in nmd mice.

Discussion

We have studied the electromyographic properties of the nmd mice and tested the efficacy of an agonistic monoclonal antibody for trkC receptors (Mab2256) on the clinical and electrophysiological progression of the disease. We show that Mab2256 treatment delayed several weeks the decline of muscular strength and it led to the electrophysiological improvement of muscular function.

Neuromuscular Impairment in nmd Mice

We observed some striking changes in nmd neuromuscular function. The major defects include a severe loss of motor nerve fibres and the inability of nmd mice to maintain a normal neuromuscular transmission with repetitive nerve stimulation. Normally, with repetitive nerve stimulation, the CMAPs gradually decline in amplitude until they reach a steady level of depression after several pulses, being the maximal degree of depression directly proportional to the stimulation frequency. We have compared this physiological response in the MG of nmd and wild-type and found that nmd mice presented a much more severe depression in CMAP amplitudes than the littermate controls, which is consistent with their clinical weakness and it is a common finding in other mouse models of motor neuron impairment (18). It would be of interest in the future to check if this phenomenon in nmd mice is due to a defect at the presynaptic terminal, a reduction of postsynaptic efficacy or a shift of the muscular fibres to a more fatigable phenotype.

CMAP mean amplitude was reduced by more than 50% in nmd mice MG on P70. This is in agreement with the reduction to 41% of lumbar motor neurons at 5 weeks described previously in this mutant (11). At later stages of the disease (P150-230), the number of motor units remaining in the MG muscle was reduced to a 35% of the control value, which is not far from the previous estimation of 28% motor neurons remaining in the lumbar spinal cord at 12-14 weeks (11). The giant motor unit potentials had a high threshold of activation so they may recruit poorly. The severe loss of motor units, together with the inability to maintain effective transmission with repetitive stimuli, explains the diminished muscular strength of these animals.

Therapeutic Potential of Neurotrophic Factors

Neurotrophic factors support survival of spinal motor neurons and have been shown to have a positive effect on alleviating the pathological symptoms in animal models of motor neuron diseases (14, 19, 20). Based of these results, recombinant neurotrophic factors have been considered for more than a decade as potential therapeutic drugs for motor neuron diseases. However, clinical trials had encountered problems such as inadequate dosage, side effects, etc (21, 22). Agonist monoclonal antibodies for neurotrophin receptors, have several theoretical advantages over exogenous administrated neurotrophic factors, e.g. their specificity for a given trophic receptor that may reduce side effects, and their long circulating half-life that facilitate the drug administration and the maintenance of therapeutic concentrations. Nevertheless, these molecules need to be studied and validated in animal models. For example, in some models the rescue of motor neurons by neurotrophic factors was found to be transient in nature (23, 24). In our experiments in nmd mice, Mab2256 retarded but not arrested disease progression.

It has been shown that ciliary neurotrophic factor (CNTF) and NT-3 can increase lifespan in the mouse mutant pmn (progressive motor neuronopathy) (14, 25, 26). Average life span of nmd mice has been described to be 54 days (10). In our animal facility, untreated nmd mice median survival was similar (64 days). Treatment of nmd mice with Mab2256 from the 3^{rd} to the 11^{th} postnatal week did not increase significantly the survival probability (median: 69 days). This result is in accordance with the fact that neuronal expression of full length IGHMBP2 in nmd mice also could not improve their survival (10).

A possible explanation of the transient effects of Mab2256 is that the relative high levels in plasma of the drug reached by some animals might have produced down regulation of trkC receptors, as has been shown in other studies with neurotrophins (27, 28). If this is the case, therapeutic dosage and dosing frequency should be adjusted to avoid this effect.

Alternatively, Mab2256 was less potent (˜3000-fold difference in the EC₅₀) and less effective (˜3 fold difference in the maximal effect) than NT-3 in the neuronal survival bioassay. Thus a higher affinity/activity version of the Mab2256 antibody might be required to achieve a longer and greater efficacy in the nmd mouse model.

Furthermore, lower motoneurons express both trkB and trkC receptors. Activation of these and perhaps other neurotrophin receptors may be necessary for clinically beneficial outcomes in the nmd mice. In the future, it will be important to test if the application of both trkB and trkC agonists simultaneously would provide greater therapeutic benefits in this disease model. Indeed, we found that both Mab2256 and NT-4/5, an endogenous trkB agonist, can restore aspects of the electrophysiological properties of the neuromuscular junction, such as in the repeated stimulation protocol, of the nmd mice. Combination of NT-3 with other neurotrophic factors may be also suitable; for example, co-injection of adenovirus vectors encoding the CNTF gene and the NT-3 gene into skeletal muscle cells of pmn mice produces a large increase in axonal survival than either vector alone (26, 29).

We do not yet know the exact cellular target(s) responsible for the positive effects seen in vivo with Mab2256 in nmd mice, but they may be motor neurons, muscle cells, glia cells or a combination of the above. Motor neurons express the NT-3 receptor trkC and respond to NT-3 with increasing survival (15, 30-32). Recent evidences suggest that NT-3, besides its effect on the survival of motor neurons, may influence the efficacy of neuromuscular transmission (33-36). Exogenous BNDF or NT-3 (but not nerve growth factor, NGF) potentiate both spontaneous and impulse-evoked synaptic activity of developing neuromuscular synapses in culture, an effect that seems to be mediated by the trkC receptor and persists as long as the factor is present (37). Furthermore, treatment of isolated neurons with NT-3 for two days increases the average sizes of quantal ACh packets at newly formed nerve-muscle synapses, whereas treatment with antibody against NT-3 or with K252a, a specific inhibitor of tyrosine kinase receptors, decreases the quantal size at existing synapses, which suggests that NT-3 may be responsible for the development and maintenance of the quantal packets (34). Enhancement of synaptic transmission by NT-3 has also been reported in adult hippocampus slices (38). NT-3 and NT-4/5 synthesized by the muscle may act in a retrograde manner on presynaptic motor neurons, thereby affecting the continued functional differentiation of the neurons by, for example, increasing the synthesis of ACh and neuregulin (39). Besides these presynaptic effects, muscle-secreted neurotrophins (BDNF, NT-3, NT-4/5 and GNDF (15, 40-43) may act on the muscle fibres themselves in an autocrine manner. Exogenous administration of NT-3 has been shown to restore NMJ architecture in curare-treated muscles previously altered by the treatment (44). The release of NT-3 from muscle cells seems to be regulated, in turn, by the synaptic activity at the NMJ (36).

Adenovirus-mediated gene transfer of NT-3 promotes terminal sprouting of motor fibres in pmn mice what suggests that NT-3 is also involved in the maintenance and regeneration of distal axon structures (26, 29). Additionally, neurotrophic factors may also influence the axonal transport from motor nerves to the spinal cord. In the pmn mice, in which motor axons degenerate due to altered tubulin assembly (45), retrograde transport of fluorescent tracers either injected into the gastrocnemius muscle or applied directly onto the cut sciatic nerve can be improved by CNTF, BDNF or NT-3, but not by GDNF or NGF (46).

It is unlikely that Mab2256 can cross the intact brain blood barrier due to its large molecular size; however, NT-3, and others neurotrophins (NT-4/5, BDNF), can be retrogradely transported along the motor neuron axons to their somata (31, 42, 47, 48). It would be of great interest to know if this monoclonal antibody, once bound to trkC, could be transported retrogradely and have a direct effect on the cell body of motor neurons. The exact of site(s) and the mode of action exerted by the trkC antibody clearly await further investigation.

Diaphragm Electrical Activity

In SMARD1 patients paralysis of the diaphragm appears during the first 13 month of life (2), but in the nmd mice breathing abnormalities manifested relatively late (10). To evaluate the functional state of the diaphragm during the final stages of the disease (P150-230), we recorded the spontaneous electrical activity of the diaphragm of mutant mice in vivo. In anaesthetised mice, no difference in the mean respiratory frequency was found between control and mutants littermates but we discovered a 26% reduction in the duration of the inspiration (TI), with a concomitant decrease in the number of action potentials during each inspiratory burst what may produce a certain reduction in diaphragm strength. From our recordings we can not discern if there are non-functioning regions within the diaphragm, as it was difficult to distinguish between zones with no-electrical-activity and mispositioning of the active electrode (see methods). It has been recently described the presence of abundant myopathic changes in the diaphragm of nmd mice (11). In addition, these mice also suffer from congestive heart failure and a muscle dystrophy-like phenotype (10) what may secondarily contribute to the respiratory distress. The number of myelinated axons, however, was not reduced in the very old nmd mice (38 weeks), which is in accordance what it has been found in 14 weeks old nmd mice (11), and in agreement with the expression of a certain amount of full length functional IGHMBP2 protein (9).

In conclusion, we have found that Mab2256 treatment on nmd mice produced a significant but transient improvement of muscular strength, as well as normalization of the amount of neuromuscular depression during high frequency nerve stimulation. Further study to elucidate the mechanism of action of this effect, together with a more complete characterization of presynaptic and postsynaptic events during neurotransmission at the neuromuscular junction should help us understand the neuromuscular defect in nmd mice, the physiological role of IGHMBP2 in motor neurons and search for the rational therapeutic approach of this terrible disease.

Materials and Methods

Mice Breeding and Genotype

B6.BLKS-nmd^{2J} mice were obtained from The Jackson Laboratory. Mice heterozygous for the nmd^{2J} were intercrossed and wild-type, heterozygous and mutant mice were used for the experiments. Mice were bred and maintained in standard conditions, except that for mutants food and water were available at the floor cage level.

Mice were genotyped as described (9). Briefly, the point mutation, cause of the phenotype of the nmd mouse (homozygous for this mutation), generates a new DdeI restriction site that is absent in wild-type mice. The PCR assay to identify carriers in unaffected offspring was performed with two oligonucleotide primers that amplify an 694 bp PCR product, where the mutation is, being the forward primer: 5′-GCTGGAAACGATCACATACCG-3′ and the reverse primer: 5′-AGCTCCTGATGATCCAATGG-3′.

Mice Treatment

Random groups of coded littermates mice of both sexes were injected intraperitoneally either with the monoclonal antibody Mab2256 (5 mg/kg body weight, two times per week, from 20-21 days of age; Rinat Neuroscience, Palo Alto, Calif.), human recombinant NT-4/5 (5 mg/kg body weight, two times per week, from 21 days of age; Genentech, San Francisco, Calif.) or PBS. All animal manipulations were performed in accordance with institutional guidelines and permissions.

Receptor Tyrosine Phosphorylation Assay

The agonist activity of Mab2256 was evaluated in a cell-based trkC receptor tyrosine phosphorylation assay as previously described (16). The half maximum effective concentrations were estimated by non-linear curve fitting using the Prism Software (GraphPad, San Diego).

Embryonic Trigeminal Neuron Survival Assay

Dissociated cultures of the trigeminal neurons were established from E12 Sprague Dawley rats. Dissected ganglia were trypsinized and dissociated by trituration (49). The neurons were plated at a low density in 96 well tissue culture plates in a defined, serum-free medium on a polyornithine/laminin substratum. NT3 and the Mab2256 antibody, at varying concentrations, were added to the cultures at the time of plating in triplicates and in quadruplicates, respectively. To quantify neuronal survival under each of the different conditions, the numbers of the neurons that survived at 48 hours after plating were counted. The half maximum effective concentrations were estimated by non-linear curve fitting using the Prism Software (GraphPad, San Diego)

Pharmacokinetic Study

Adult female CD-1 mice (n=3) were injected intraperitoneally with Mab2256 at 2 mg/kg. The animals were then bled subsequently at 24, 48, 136 and 184 hours post-injection. The serum concentration of Mab2256 at different time points were determined as described below.

Determination of Monoclonal Antibody Concentrations

The serum level of mouse monoclonal antibody Mab2256 was determined by a standard sandwich ELISA. The 96 well Maxisorp plate (Nunc) was preabsorbed overnight at 4° C. with 0.2 mg/mL of a protein A column-purified, recombinant human trkC extracellular domain-IgG Fc fusion protein (Rinat Neuroscience) expressed by transient transfection of HEK293 cells. The trkC coated plate was then blocked at 25° C. for 1 hour with phosphate buffered saline (PBS) with 0.5% bovine serum albumin and 0.05% Tween-20. The plate was washed 3 times in PBS with 0.05% Tween-20. The standard dilution series of Mab2256 as well as appropriately diluted serum samples were incubated in the plate at 25° C. for 1 hour, followed by 3 washes. The horseradish peroxidase (HRP)-conjugated rabbit anti-mouse IgG (Jackson ImmunoResearch) was applied at 1:5000, incubated at 25° C. for 1 hour to detect the bound mouse monoclonal antibody. Finally the signals were detected by colorimetric reaction of HRP with TMB substrate (KPL, Denmark).

Neurological Tests

Mutant and littermate control mice were subjected to standard neurological examination to quantify the onset and extension of the neuromuscular defect (SHIRPA protocol; www.mgu.har.mrc.ac.uk/mutabase/shirpa_summary.html) (50). For balance measurements mice were lowered onto a square thin stick by tail suspension, and allowed to stand on top. The stick was then rotated each second by hand for 10 seconds, and the ability of the mice to remain on the stick was measured in seconds.

For fore limb grip strength, mice were held above a horizontal wire and lowered to allow the fore limbs to grip the wire The ability of the mice to remain attached by the fore limbs was scored during 10 seconds.

Electromyography (EMG)

Intramuscular compound action potentials (CMAP) were recorded as previously described (18) from anaesthetized (100 mg/Kg Ketamine+10 mg/Kg Xylazine) wild-type and nmd mice at P70. Briefly, recording needle electrodes were placed either into the dorsal foot muscles with a reference electrode on the base of the 5^{th} phalanx or into the medial part of the gastrocnemius (MG). A ground electrode was placed at the base of the tail. Stimulating needle electrodes were placed at the sciatic notch and the head of the fibula. Stimulation protocols of supramaximal current pulses (0.05 ms duration, 5 mA amplitude) were applied either as single pulses or as short duration train of pulses of 10, 20, 50 and 100 Hz. Stimulation pulses were generated by an isolated pulse stimulator (A-M Systems, Model 2100). Recorded outputs were differentially amplified (Brownlee Precision, Model 210A), digitally acquired at 20,000 samples/sec (ADInstruments, PowerLab/4SP) and stored in a computer for later analysis.

Motor unit number estimation (MUNE) from the MG was calculated by dividing the averaged size of a single motor unit potential into a maximal CMAP that represents the sum of all motor units. Sampling of single motor unit potentials were done by the incremental method (51, 52) that consists in the application of finely controlled current in very small steps from sub threshold levels until the progressive recruitment of ten responses. Each current amplitude was applied three times and was considered stable, and therefore accepted, if they were identical. Individual motor unit amplitudes were obtained by subtracting amplitudes to each response to that of the previous response. The average of the individual values gave us an estimation of the single motor unit action potential (SMUAP) size.

In vivo diaphragmatic recordings were performed by inserting a needle electrode behind the xyphoid process slightly off middle line to either site. The reference electrode was place on the chest and the ground electrode at the base of the tail. The diaphragm was readily identified by rhythmical burst discharges synchronous with respiration. The inspiratory discharges were quantified by the peak amplitude and area of the integral of the recording. Inspiratory durations (T_{I}) were also analysed. Averaged values were calculated from six consecutives breathing cycles.

Studies were performed with coded mice so that the electromyographer was blinded as to which mice were being tested.

All data are reported as mean±SEM. Statistical significance was evaluated using a Student's t-test. The criterion level for determination of statistical significance was set at P<0.05 for all experiments.

Histology

Mice were sacrificed with an overdose of Ketamine/Xylazine and right side phrenic nerves were obtained close to their entry into the diaphragm muscle. Nerves were fixed with 4% paraformaldehyde and 2.5% glutaraldehyde fixative in PBS, and then post-fixed with 2% osmium tetroxide and embedded in spurr resine (plastic embedding). Sections of 2 μm thick were stained with toluidine blue and examined by light microscopy (Axiovert 35, Zeiss) Myelinated axons were counted for each nerve.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention, which is delineated by the appended claims.

### REFERENCES

1. Lefebvre, S., Burglen, L., Reboullet, S., Clermont, O., Burlet, P., Viollet, L., Benichou, B., Cruaud, C., Millasseau, P., Zeviani, M. et al. (1995) Identification and characterization of a spinal muscular atrophy-determining gene. Cell, 80, 155-65.

2. Grohmann, K., Wienker, T. F., Saar, K., Rudnik-Schoneborn, S., Stoltenburg-Didinger, G., Rossi, R., Novelli, G., Nurnberg, G., Pfeufer, A., Wirth, B. et al. (1999) Diaphragmatic spinal muscular atrophy with respiratory distress is heterogeneous, and one form Is linked to chromosome 11q13-q21. Am J Hum Genet, 65, 1459-62.

3. Grohmann, K., Schuelke, M., Diers, A., Hoffmann, K., Lucke, B., Adams, C., Bertini, E., Leonhardt-Horti, H., Muntoni, F., Ouvrier, R. et al. (2001) Mutations in the gene encoding immunoglobulin mu-binding protein 2 cause spinal muscular atrophy with respiratory distress type 1. Nat Genet, 29, 75-7.

4. Grohmann, K., Varon, R., Stolz, P., Schuelke, M., Janetzki, C., Bertini, E., Bushby, K., Muntoni, F., Ouvrier, R., Van Maldergem, L. et al. (2003) Infantile spinal muscular atrophy with respiratory distress type 1 (SMARD1). Ann Neurol, 54, 719-24.

5. Pitt, M., Houlden, H., Jacobs, J., Mok, Q., Harding, B., Reilly, M. and Surtees, R. (2003) Severe infantile neuropathy with diaphragmatic weakness and its relationship to SMARD1. Brain, 126, 2682-92.

6. Wirth, B. (2000) An update of the mutation spectrum of the survival motor neuron gene (SMN1) in autosomal recessive spinal muscular atrophy (SMA). Hum Mutat, 15, 228-37.

7. Mercuri, E., Bertini, E., Messina, S., Pelliccioni, M., D'Amico, A., Colitto, F., Mirabella, M., Tiziano, F. D., Vitali, T., Angelozzi, C. et al. (2004) Pilot trial of phenylbutyrate in spinal muscular atrophy. Neuromuscul Disord, 14, 130-5.

8. Cook, S. A., Johnson, K. R., Bronson, R. T. and Davisson, M. T. (1995) Neuromuscular degeneration (nmd): a mutation on mouse chromosome 19 that causes motor neuron degeneration. Mamm Genome, 6, 187-91.

9. Cox, G. A., Mahaffey, C. L. and Frankel, W. N. (1998) Identification of the mouse neuromuscular degeneration gene and mapping of a second site suppressor allele. Neuron, 21, 1327-37.

10. Maddatu, T. P., Garvey, S. M., Schroeder, D. G., Hampton, T. G. and Cox, G. A. (2004) Transgenic rescue of neurogenic atrophy in the nmd mouse reveals a role for Ighmbp2 in dilated cardiomyopathy. Hum Mol Genet, 13, 1105-15.

11. Grohmann, K., Rossoll, W., Kobsar, I., Holtmann, B., Jablonka, S., Wessig, C., Stoltenburg-Didinger, G., Fischer, U., Hubner, C., Martini, R. et al. (2004) Characterization of Ighmbp2 in motor neurons and implications for the pathomechanism in a mouse model of human spinal muscular atrophy with respiratory distress type 1 (SMARD1). Hum Mol Genet.

12. Arakawa, Y., Sendtner, M. and Thoenen, H. (1990) Survival effect of ciliary neurotrophic factor (CNTF) on chick embryonic motoneurons in culture: comparison with other neurotrophic factors and cytokines. J. Neurosci, 10, 3507-15.

13. Sendtner, M., Kreutzberg, G. W. and Thoenen, H. (1990) Ciliary neurotrophic factor prevents the degeneration of motor neurons after axotomy. Nature, 345, 440-1.

14. Sendtner, M., Schmalbruch, H., Stockli, K. A., Carroll, P., Kreutzberg, G. W. and Thoenen, H. (1992) Ciliary neurotrophic factor prevents degeneration of motor neurons in mouse mutant progressive motor neuronopathy. Nature, 358, 502-4.

15. Henderson, C. E., Camu, W., Mettling, C., Gouin, A., Poulsen, K., Karihaloo, M., Rullamas, J., Evans, T., McMahon, S. B., Armanini, M. P. et al. (1993) Neurotrophins promote motor neuron survival and are present in embryonic limb bud. Nature, 363, 266-70.

16. Sadick, M. D., Galloway, A., Shelton, D., Hale, V., Weck, S., Anicetti, V. and Wong, W. L. (1997) Analysis of neurotrophin/receptor interactions with a gD-flag-modified quantitative kinase receptor activation (gD.KIRA) enzyme-linked immunosorbent assay. Exp Cell Res, 234, 354-61.

17. Poduslo, J. F. and Curran, G. L. (1996) Permeability at the blood-brain and blood-nerve barriers of the neurotrophic factors: NGF, CNTF, NT-3, BDNF. Brain Res Mol Brain Res, 36, 280-6.

18. Fernandez-Chacon, R., Wolfel, M., Nishimune, H., Tabares, L., Schmitz, F., Castellano-Munoz, M., Rosenmund, C., Montesinos, M. L., Sanes, J. R., Schneggenburger, R. et al. (2004) The synaptic vesicle protein CSP alpha prevents presynaptic degeneration. Neuron, 42, 237-51.

19. Sagot, Y., Vejsada, R. and Kato, A. C. (1997) Clinical and molecular aspects of motoneurone diseases: animal models, neurotrophic factors and Bcl-2 oncoprotein. Trends Pharmacol Sci, 18, 330-7.

20. Mitsumoto, H., Ikeda, K., Klinkosz, B., Cedarbaum, J. M., Wong, V. and Lindsay, R. M. (1994) Arrest of motor neuron disease in wobbler mice cotreated with CNTF and BDNF. Science, 265, 1107-10.

21. Miller, R. G., Petajan, J. H., Bryan, W. W., Armon, C., Barohn, R. J., Goodpasture, J. C., Hoagland, R. J., Parry, G. J., Ross, M. A. and Stromatt, S. C. (1996) A placebo-controlled trial of recombinant human ciliary neurotrophic (rhCNTF) factor in amyotrophic lateral sclerosis. rhCNTF ALS Study Group. Ann Neurol, 39, 256-60.

22. Thoenen, H. and Sendtner, M. (2002) Neurotrophins: from enthusiastic expectations through sobering experiences to rational therapeutic approaches. Nat Neurosci, 5 Suppl, 1046-50.

23. Diener, P. S. and Bregman, B. S. (1994) Neurotrophic factors prevent the death of CNS neurons after spinal cord lesions in newborn rats. Neuroreport, 5, 1913-7.

24. Vejsada, R., Sagot, Y. and Kato, A. C. (1995) Quantitative comparison of the transient rescue effects of neurotrophic factors on axotomized motoneurons in vivo. Eur J Neurosci, 7, 108-15.

25. Sagot, Y., Tan, S. A., Baetge, E., Schmalbruch, H., Kato, A. C. and Aebischer, P. (1995) Polymer encapsulated cell lines genetically engineered to release ciliary neurotrophic factor can slow down progressive motor neuronopathy in the mouse. Eur J Neurosci, 7, 1313-22.

26. Haase, G., Kennel, P., Pettmann, B., Vigne, E., Akli, S., Revah, F., Schmalbruch, H. and Kahn, A. (1997) Gene therapy of murine motor neuron disease using adenoviral vectors for neurotrophic factors. Nat Med, 3, 429-36.

27. Bibel, M. and Barde, Y. A. (2000) Neurotrophins: key regulators of cell fate and cell shape in the vertebrate nervous system. Genes Dev, 14, 2919-37.

28. Knusel, B., Gao, H., Okazaki, T., Yoshida, T., Mori, N., Hefti, F. and Kaplan, D. R. (1997) Ligand-induced down-regulation of Trk messenger RNA, protein and tyrosine phosphorylation in rat cortical neurons. Neuroscience, 78, 851-62.

29. Sendtner, M. (1997) Gene therapy for motor neuron disease. Nat Med, 3, 380-1.

30. Oppenheim, R. W. (1996) Neurotrophic survival molecules for motoneurons: an embarrassment of riches. Neuron, 17, 195-7.

31. Yan, Q., Elliott, J. L., Matheson, C., Sun, J., Zhang, L., Mu, X., Rex, K. L. and Snider, W. D. (1993) Influences of neurotrophins on mammalian motoneurons in vivo. J Neurobiol, 24, 1555-77.

32. Duberley, R. M., Johnson, I. P., Anand, P., Leigh, P. N. and Cairns, N. J. (1997) Neurotrophin-3-like immunoreactivity and Trk C expression in human spinal motoneurones in amyotrophic lateral sclerosis. J Neurol Sci, 148, 33-40.

33. Wang, T., Xie, K. and Lu, B. (1995) Neurotrophins promote maturation of developing neuromuscular synapses. J Neurosci, 15, 4796-805.

34. Liou, J. C. and Fu, W. M. (1997) Regulation of quantal secretion from developing motoneurons by postsynaptic activity-dependent release of NT-3. J Neurosci, 17, 2459-68.

35. Sendtner, M. (1998) Neurotrophic factors: effects in modulating properties of the neuromuscular endplate. Cytokine Growth Factor Rev, 9, 1-7.

36. Poo, M. M. (2001) Neurotrophins as synaptic modulators. Nat Rev Neurosci, 2, 24-32.

37. Lohof, A. M., Ip, N. Y. and Poo, M. M. (1993) Potentiation of developing neuromuscular synapses by the neurotrophins NT-3 and BDNF. Nature, 363, 350-3.

38. Kang, H. and Schuman, E. M. (1995) Long-lasting neurotrophin-induced enhancement of synaptic transmission in the adult hippocampus. Science, 267, 1658-62.

39. Loeb, J. A. and Fischbach, G. D. (1997) Neurotrophic factors increase neuregulin expression in embryonic ventral spinal cord neurons. J Neurosci, 17, 1416-24.

40. Griesbeck, O., Parsadanian, A. S., Sendtner, M. and Thoenen, H. (1995) Expression of neurotrophins in skeletal muscle: quantitative comparison and significance for motoneuron survival and maintenance of function. J Neurosci Res, 42, 21-33.

41. Henderson, C. E., Phillips, H. S., Pollock, R. A., Davies, A. M., Lemeulle, C., Armanini, M., Simmons, L., Moffet, B., Vandlen, R. A., Simpson, L. C. et al. (1994) GDNF: a potent survival factor for motoneurons present in peripheral nerve and muscle. Science, 266, 1062-4.

42. Koliatsos, V. E., Clatterbuck, R. E., Winslow, J. W., Cayouette, M. H. and Price, D. L. (1993) Evidence that brain-derived neurotrophic factor is a trophic factor for motor neurons in vivo. Neuron, 10, 359-67.

43. Funakoshi, H., Frisen, J., Barbany, G., Timmusk, T., Zachrisson, O., Verge, V. M. and Persson, H. (1993) Differential expression of mRNAs for neurotrophins and their receptors after axotomy of the sciatic nerve. J Cell Biol, 123, 455-65.

44. Loeb, J. A., Hmadcha, A., Fischbach, G. D., Land, S. J. and Zakarian, V. L. (2002) Neuregulin expression at neuromuscular synapses is modulated by synaptic activity and neurotrophic factors. J Neurosci, 22, 2206-14.

45. Bommel, H., Xie, G., Rossoll, W., Wiese, S., Jablonka, S., Boehm, T. and Sendtner, M. (2002) Missense mutation in the tubulin-specific chaperone E (Tbce) gene in the mouse mutant progressive motor neuronopathy, a model of human motoneuron disease. J Cell Biol, 159, 563-9.

46. Sagot, Y., Rosse, T., Vejsada, R., Perrelet, D. and Kato, A. C. (1998) Differential effects of neurotrophic factors on motoneuron retrograde labeling in a murine model of motoneuron disease. J Neurosci, 18, 1132-41.

47. DiStefano, P. S., Friedman, B., Radziejewski, C., Alexander, C., Boland, P., Schick, C. M., Lindsay, R. M. and Wiegand, S. J. (1992) The neurotrophins BDNF, NT-3, and NGF display distinct patterns of retrograde axonal transport in peripheral and central neurons. Neuron, 8, 983-93.

48. Yan, Q., Elliott, J. and Snider, W. D. (1992) Brain-derived neurotrophic factor rescues spinal motor neurons from axotomy-induced cell death. Nature, 360, 753-5.

49. Davies, A. M., Horton, A., Burton, L. E., Schmelzer, C., Vandlen, R. and Rosenthal, A. (1993) Neurotrophin-4/5 is a mammalian-specific survival factor for distinct populations of sensory neurons. J Neurosci, 13, 4961-7.

50. Rogers, S. D., Demaster, E., Catton, M., Ghilardi, J. R., Levin, L. A., Maggio, J. E. and Mantyh, P. W. (1997) Expression of endothelin-B receptors by glia in vivo is increased after CNS injury in rats, rabbits, and humans. Exp Neurol, 145, 180-95.

51. McComas, A. J. (1991) Motor unit estimation: methods, results, and present status. Muscle Nerve, 14, 585-97.

52. Shefner, J. M. and Gooch, C. L. (2002) Motor unit number estimation in neurologic disease. Adv Neurol, 88, 33-52.

53. Ruiz, R., Lin, J., Forgie, A., Foletti, D., Shelton, D., Rosenthal, A. and Tabares, L. (2005) Treatment with trkC agonist antibodies delays disease progression in neuromuscular degeneration (nmd) mice. Human Molecular Genetics, 14, 1825-1837.

### ABBREVIATIONS

BDNF: brain derived neurotrophic factor

bpm: breathes per minute

CMAP: compound muscular action potential

CNTF: ciliary neurotrophic factor

GDNF: glial derived neurotrophic factor

MG: medial gastrocnemius

IGHMBP2: immunoglobulin μ-binding protein 2 gene

nmd: neuromuscular degeneration

MUNE: motor unit number estimation

NGF: nerve grow factor

NMJ: neuromuscular junction

NT-3: neurotophin 3

NT-4/5: neurotrophin 4/5

SMA: spinal muscular atrophy

SMARD1: spinal muscular atrophy type 1

SMN: survival motor neuron gene

SMUAP: single motor unit action potential

TI: time of inspiration

trkA: tyrosine kinase receptor A

trkB: tyrosine kinase receptor B

trkC: tyrosine kinase receptor C

What is claimed is:

1. A method for treating a lower motor neuron disease in an individual comprising administering to the individual an effective amount of an agonist anti-trkC antibody.

2. The method of claim 1, wherein the lower motor neuron disease is spinal muscular atrophy (SMA).

3. The method of claim 1, wherein the lower motor neuron disease is spinal muscular atrophy with respiratory distress type 1 (SMARD1).

4. The method of claim 3, wherein the muscle strength in the individual is improved after administering the agonist trkC antibody.

5. The method of claim 1, wherein the agonist anti-trkC antibody binds human trkC.

6. The method of claim 1, wherein the agonist anti-trkC antibody binds human trkC with an affinity of less than about 1 nM as assessed by the Fab fragment of the agonist anti-trkC antibody.

7. The method of claim 1, wherein the agonist anti-trkC antibody binds human trkC and rodent trkC.

8. The method of claim 1, wherein the agonist anti-trkC antibody binds an epitope in domain 4 of trk C.

9. The method of claim 1, wherein the agonist anti-trkC antibody is a human antibody.

10. The method of claim 1, wherein the agonist anti-trkC antibody is a humanized antibody

11. The method of claim 1, wherein the agonist anti-trkC antibody is a monoclonal antibody.

12. The method of claim 1, wherein the agonist anti-trkC antibody comprising a heavy chain variable region comprising three CDRs from SEQ ID NO:1, and a light chain variable region comprising three CDRs from SEQ ID NO:2.

13. The method of claim 1, wherein the agonist anti-trkC antibody comprises the amino acid sequence of the heavy chain variable region shown in SEQ ID NO:1, and the amino acid sequence of the light chain variable region shown in SEQ ID NO:2.

14. The method of claim 1, wherein the agonist anti-trkC antibody is antibody A5.

15. A pharmaceutical composition for treating a lower motor neuron disease in an individual, comprising an agonist anti-trkC antibody and a pharmaceutically acceptable carrier.

16. A kit comprising:
a) a pharmaceutical composition comprising an agonist anti-trkC antibody and a pharmaceutically acceptable carrier; and
b) instructions for administering an effective amount of said pharmaceutical composition to an individual for treating a lower motor neuron disease.
